(19) United States
(12) Patent Application Publication
(10) Pub. No.: US 2023/0054093 A1
(43) Pub. Date: Feb. 23, 2023

(54) TREATMENT

(71) Applicant: Pneumagen Ltd, Fife (GB)

(72) Inventors: Helen Connaris, St. Andrews (GB); Lei Yang, Fife (GB); Jane Alexandra Potter, Fife (GB)

(21) Appl. No.: 17/753,273

(22) PCT Filed: Aug. 28, 2020
(86) PCT No.: PCT/EP2020/074140

§ 371 Date: Feb. 25, 2022

Related U.S. Application Data
(60) Provisional application No. 62/892,750, filed on Aug. 28, 2019.

(30) Foreign Application Priority Data
Aug. 28, 2019 (GB) 1912365.2

(51) Int. Cl. A61K 38/04; A61P 29/00; A61K 9/00

(52) U.S. Cl. CPC A61K 38/04; A61P 29/00; A61K 9/0043

(57) ABSTRACT

Disclosed are molecules with affinity for (or an ability to bind to) sialic acid (and in particular sialoglycoconjugates) on cell surfaces (these including sialic acid containing glycoproteins and cell surface sialic acid receptors), for use in treating or preventing of inflammatory diseases and/or conditions with an inflammatory aeitiology. The disclosed molecules may be of particular use in the treatment and/or prevention of diseases and/or conditions characterised by inflammation occurring as a consequence of a cascade of cytokines, inflammation that may occur in and around the tissues and/or structures of the lung and inflammation occurring as a result of an infection.

### RELATED APPLICATIONS

This application is a 35 U.S.C. § 371 national stage of PCT Application No. PCT/EP2020/074140, filed on Aug. 28, 2020, which claims priority from United Kingdom Patent Application No. 1912365.2, filed on Aug. 28, 2019, and to U.S. Provisional Patent Application No. 62/892,750, filed Aug. 28, 2019, the contents of each of which are incorporated herein by reference. The above-referenced PCT International Application was published in the English language as International Publication No. WO 2021/038080 A1 on Mar. 4, 2021.

### STATEMENT REGARDING ELECTRONIC FILING OF A SEQUENCE LISTING

A Sequence Listing in ASCII text format, submitted under 37 C.F.R. § 1.821, entitled RpI-9013-177_ST25.bd, 52,431 bytes in size, generated on Sep. 6, 2022, and filed via EFS-Web, is provided in lieu of a paper copy. This Sequence Listing is hereby incorporated by reference into the specification for its disclosures.

### FIELD OF THE INVENTION

The invention provides molecules for use in compositions, medicaments and methods for the treatment or prevention of lung inflammatory diseases, pneumonia and/or bronchitis.

### BACKGROUND OF THE INVENTION

Inflammation and exaggerated cytokine/chemokine responses within the lung can lead to a number of damaging pathologies. For example, infection can result in inflammation which causes tissue damage and other complications. Pneumonia and bronchitis are two such complications which can occur as a consequence of viral, bacterial and/or fungal infection. Pneumonia and bronchitis can also occur as a result of ventilation within the intensive care setting and inhalation of toxic substances and chemicals.

In the age of antibiotic resistance there is a need for alternate medicaments, compositions and methods which can be used to prevent or treat lung inflammatory diseases

### SUMMARY OF THE INVENTION

The present disclosure is based on the finding that molecules with affinity for (or an ability to bind to) sialic acid (and in particular sialoglycoconjugates) on cell surfaces (these including sialic acid containing glycoproteins and cell surface sialic acid receptors), find utility in the treatment and/or prevention of inflammatory diseases and/or conditions with an inflammatory aeitiology. Diseases and/or conditions of this type may include, for example, those diseases and/or conditions characterised by inflammation occurring as a consequence of a cascade of cytokines.

The various molecules described herein may be for use in methods of treating or preventing inflammation that may occur in and around the tissues and/or structures of the lung.

Inflammation in the lung may occur as a result of an infection. As such, the molecules of this disclosure may be used to treat or prevent inflammation that occurs as a result of a microbial, for example, viral, bacterial and/or fungal infection in the lung. One of skill will appreciate that infections of this type can induce potentially harmful cytokine and/or chemokine cascades that lead cell influx (swelling) and tissue damage.

An infection of the lung may lead to a form of bronchitis, bronchiolitis or pneumonia. Accordingly, diseases and/or conditions which may be treated or prevented using the various sialic acid binding molecules described herein may include, for example those selected from the group consisting of:
(i) (chronic and acute) pneumonia;
(ii) (chronic and acute) bronchitis; and
(iii) (chronic and acute) bronchiolitis.

More specifically, the molecules described herein may be used to treat or prevent one or more of the following types or forms of pneumonia:
(a) bacterial pneumonia: this can occur as a consequence of a pneumococcal infection. For example infections involving Streptococcus pneumoniae, Haemophilus influenzae and Staphylococcus aureus, can all result in pneumonia;
(b) viral pneumonia—frequently caused by the respiratory syncytial virus (RSV) and sometimes influenza type A or B; and
(c) fungal pneumonia—can occur in subjects who are immunocompromised. Fungal pneumonia may be the complications following an Aspergillosis infection.

Another category of pneumonia which can be treated or prevented by a molecule described herein is “aspiration pneumonia”—this being a pneumonia caused by breathing in vomit, a foreign object or particle and/or a harmful or toxic substance, such as smoke or a chemical.

Other forms of pneumonia which can be treated using a molecule described herein may include, for example those pneumonias which occur in subjects within the hospital or care facility setting, often while said subject is being treated for another, unrelated condition. For example, subjects (or patients) in intensive care on breathing machines are particularly at risk of developing ventilator-associated pneumonia. Accordingly, a molecule of this invention may be used, perhaps prophylactically, to ensure that the risk of pneumonia is reduced in subjects who are vulnerable/predisposed to pneumonia (this would include patients or subjects with underlying health issues, intensive care patients (particularly those who are being ventilated) and/or immunocompromised patients).

Other diseases and/or conditions, including those without a microbial aeitiology, may also result in some level of inflammation in the lung. Diseases and/or conditions of this type can be treated and/or prevented using any of the molecules described herein and may include (for example) one or more of the diseases listed below:
(i) chronic obstructive pulmonary disease (COPD);
(ii) asthma;
(iii) emphysema; and
(iv) interstitial lung disease.

For convenience, all of the diseases and conditions described herein shall be referred to under the term “lung inflammatory disease”.

The present disclosure provides a sialic acid binding molecule for use in the treatment and/or prevention of lung inflammatory disease.

Further provided is the use of a sialic acid binding molecule in the manufacture of a medicament for use in the treatment and/or prevention of lung inflammatory disease.

The disclosure also provides a method of treating or preventing a lung inflammatory disease, said method comprising administering a subject in need thereof a therapeutically effective amount of a sialic acid binding molecule.

The disclosure also provides sialic acid binding molecules and medicaments and methods comprising sialic acid binding molecules, for use in methods of treating or preventing a lung inflammatory disease.

Throughout this specification, the terms “comprise”, “comprising” and/or “comprises” is/are used to denote aspects and embodiments of this invention that “comprise” a particular feature or features. It should be understood that this/these terms may also encompass aspects and/or embodiments which “consist essentially of” or “consist of” the relevant feature or features.

Without wishing to be bound by theory, it is suggested that when administered to a subject, a sialic acid binding molecule of this disclosure can suppress, inhibit or reduce a cytokine response within the lung and/or suppress, inhibit or reduce the influx of cells, including, immune cells, into the lung tissue. Combined, these actions result in a reduced inflammatory response within the lung and also reduce any damage typically associated with exaggerated cytokine/chemokine cascades.

While it may have been established that certain sialic acid binding proteins can be used to prime or modulate the immune system by increasing the levels of certain cytokines (including various proinflammatory cytokines), and that a primed or modulated immune response may impact on the pathology of a whole host of different pathogens (including those that do not bind or which do not primarily bind sialic acid during pathogenesis), one of skill would still not have been led to the finding that the sialic acid binding molecules described herein inhibit cytokine responses and cell migration and can therefore be used to treat or prevent diseases or conditions which are caused or contributed to by activated cytokines and increased cell migration.

Further, for prior art disclosures where sialic acid binding molecules have been used as agents capable of blocking the binding of pathogens to cell surface sialic acid/sialoglycoconjugates, the utility of the sialic acid binding molecule is rooted in the fact that both the sialic acid binding molecule and the pathogen bind sialic acid; this is not the case here. Many of the pathogens or clinical situations that lead to lung inflammatory diseases of the type described herein, do not concern or involve binding between the pathogen and sialic acid. As stated, the present disclosure is based on the unexpected finding that certain sialic acid binding molecules act to inhibit specific cytokines (including proinflammatory cytokines) and cell migration.

The findings reported in this disclosure have important implications for the formulation and administration of molecules with sialic acid binding affinity and for the subsequent use of these formulations in the treatment and/or prevention of lung inflammatory diseases.

For example, the finding that the sialic acid binding molecules described herein can be used to treat or prevent a lung inflammatory disease allows for the use or preparation of the disclosed sialic acid binding molecules as formulations suitable for mucosal, intranasal or inhalation administration.

Accordingly, the disclosure provides a composition for mucosal administration, said composition comprising a sialic acid binding molecule for use in the treatment and/or prevention of lung inflammatory diseases.

It should be noted that the term “mucosal administration” embraces compositions that have been formulated for administration to any mucosal surface, including, for example, respiratory surfaces, nasal passages and the like. The term also embraces compositions formulated for administration by inhalation. Compositions suitable (or formulated) for mucosal administration may include compositions, which are intended to be administered intranasally (or nasally).

Thus a composition for mucosal administration may be formulated with excipients, diluents and/or buffers which are suitable for use in any type of mucosal administration as described above.

Compositions for mucosal (for example intranasal) administration may comprise solutions of the sialic acid binding molecule(s) to be administered and/or particles (comprising the same) for aerosol dispersion or dispensed in drinking water. When dispensed such compositions should desirably have a particle diameter in the range 10 to 200 microns to enable retention in, for example, the nasal cavity; this may be achieved by, as appropriate, use of a powder of a suitable particle size or choice of an appropriate valve. Other suitable compositions include coarse powders having a particle diameter in the range 20 to 500 microns, for administration by rapid inhalation through the nasal passage from a container held close up to the nose, and nasal drops comprising 0.2 to 5% w/v of an active compound in aqueous or oily solution or suspension. A composition for mucosal administration may be provided in the form of a liquid spray.

Importantly, this disclosure provides sialic acid binding molecules for prophylactic use. Specifically, the sialic acid molecules described herein may be used prophylactically in order to prevent a lung inflammatory diseases in subjects.

Accordingly, the present disclosure provides a sialic acid binding molecule for use in the prevention a lung inflammatory disease. Also provided is the use of a sialic acid binding molecule in the manufacture of a medicament for use in the prevention of a lung inflammatory disease. A method of prophylaxis or preventing a lung inflammatory disease may comprise administering a subject in need thereof a therapeutically effective amount of a sialic acid binding molecule. In all cases, the sialic acid binding molecule may be:
(i) prepared as a formulation suitable for mucosal (including intranasal) administration; and/or
(ii) administered to the subject's mucosal surfaces and/or intranasally.

Additionally, in all cases, the lung inflammatory disease may be a pneumonia, bronchiolitis and/or bronchitis.

As used herein and in any method of prophylaxis or method or sialic acid binding molecule for use in preventing a lung inflammatory disease, the term “subject” may extend to any subject predisposed, susceptible or at risk of developing a lung inflammatory disease, including pneumonia, bronchiolitis and/or bronchitis. The subject may be a neonate, an infant or a child. The subject may be an adult or elderly. The subject may be an immunocompromised subject. The subject may have one or more underlying or chronic health problems—in particular, problems affecting the respiratory tract and/or respiration. For example, the subject may suffer from asthma. The subject may have a viral, bacterial and/or fungal infection; that infection may reside within the lung of the subject. The subject may be an intensive care patient.

Throughout this specification, the term “sialic acid binding molecule” and this embraces any useful sialic acid binding molecule. Useful sialic acid binding molecules may take any form and/or belong to any class of molecule or compound (for example they may be proteins, peptides, carbohydrates, antibodies and the like) and the term “sialic acid” embraces all forms of N- or O-substituted neuraminic acid and includes all synthetic, naturally occurring and/or modified forms thereof. Sialic acids may be found as components of cell surface molecules, glycoproteins and glycolipids. Most often, sialic acids are present at the end (terminal regions) of sugar chains connected to cell membranes and/or proteins. For example, some cells of the human upper respiratory tract comprise α-2,6-linked sialic acid receptors and other cells of the upper and lower respiratory tracts comprise α-2,3-linked sialic acid receptors. The sialic acid family encompasses a number (approximately 50) of derivatives that may result from acetylation, glycolylation, lactonisation and methylation at C4, C5, C7, C8 and C9. All such derivatives are to be embraced by the term “sialic acid”.

Furthermore, sialic acids are found linked α(2,3) or α(2,6) to Gal and GaINAc or α(2,8) or α(2,9) to another sialic acid. Accordingly, it is important to understand that while the term “sialic acid” is used throughout this specification, it encompasses all derivatives, analogues or variants (either naturally occurring or synthetically generated) thereof as well as monomers, dimers, trimers, oligomers, polymers or concatamers comprising the same.

Thus, a sialic acid binding molecule of this disclosure (and for use as described herein) comprises a moiety which exhibits an affinity for sialic acid—which may include all forms of sialic acid described above and any form of sialic acid present on the surface of a cell (perhaps as part of a cell surface receptor), for example a mammalian cell. These various forms of sialic acid may be collectively referred to as “sialic acid moieties”.

The sialic acid binding molecules of this disclosure exhibit an affinity for sialic acid and as such they may bind/couple to and/or associate with one or more sialic acid moieties. Thus, the term “sialic acid binding molecule” may further encompass fragments of whole sialic acid binding molecules which retain an ability to bind to or otherwise couple or associate with a sialic acid moiety.

Sialic acid binding molecules, including those for the uses, compositions and methods described herein, may comprise a single sialic acid binding molecule (a monomeric or monovalent molecule, for example) or, alternatively, two or more sialic acid binding molecules—which two or more molecules may be the same or different—a polymeric or multivalent molecule, for example.

A sialic acid binding molecule, including those for uses, compositions and methods described herein may comprise, consist essentially of or consist of, one or more of the sialic acid binding molecules known as “carbohydrate binding modules” (CBMs). CBMs suitable for use exhibit an affinity for sialic acid. CBMs are classified into families and CBMs classed as members of the family 40 CBMs (CBM40) may be useful. The family 40 CBMs embrace molecules of approximately 200 residues and are often found at the N-terminus of GH33 sialidases. They may also be found inserted in the β-propeller of GH33 sialidases.

The disclosure may embrace the use of molecules, for example, larger molecules, which comprise a sialic acid binding component. As stated, that sialic acid binding component (i.e. the sialic acid binding molecule) may itself comprise (consist of or consist essentially of) a CBM, for example, a CBM40. By way of (non-limiting) example, the molecules (e.g. the sialic acid binding molecules) of this disclosure may not only exhibit an ability to bind sialic acid, but may also have one or more other functions. For example, the molecules may have enzymatic activity. For example, a useful molecule may comprise a CBM (as described herein) and exhibit some sialidase activity.

A useful sialic acid binding molecule may be a fusion protein comprising an enzymatic portion and a sialic acid binding portion—wherein the sialic acid binding portion comprises a sialic acid binding molecule of this disclosure. In such cases, the enzymatic portion may be fused to the sialic acid binding portion. As stated, the enzymatic portion of any useful fusion protein may comprise (or have, or exhibit) sialidase activity.

The sialic acid binding protein or CBM for the various uses, methods and compositions described herein, may not be provided as part of, or comprised within, a molecule (for example a fusion protein) with enzymatic (for example sialidase) activity. Additionally or alternatively, the sialic acid binding molecule may not (i) bind heparin or heparin sulfate and/or (ii) comprise the GAG-binding domain of a protein that binds heparin or heparin sulfate moieties.

As such, the present disclosure provides a CBM or CBM40 for use in the treatment and/or prevention of the lung inflammatory disease disclosed herein.

Further provided is the use of a CBM or CBM40 in the manufacture of a medicament for use in the treatment and/or prevention of the lung inflammatory diseases disclosed herein.

The disclosure also provides a method of treating or preventing a lung inflammatory disease of this disclosure, said method comprising administering a subject in need thereof a therapeutically effective amount of a CBM or CBM40.

The disclosure also provides sialic acid binding molecules and medicaments and methods comprising a CBM or CBM40 for use in methods of treating or preventing a lung inflammatory disease such as, for example pneumonia, bronchiolitis and/or bronchitis.

The disclosure provides a composition for mucosal administration, said composition comprising a CBM and/or CBM40 for use in the treatment and/or prevention of the lung inflammatory diseases of this disclosure. As stated, a composition for mucosal administration may be formulated with excipients, diluents and/or buffers which are suitable for use in any type of mucosal administration, including, for example intranasal administration.

The disclosure may further provide a CBM or CBM40 for prophylactic use. Specifically, CBM or CBM40 described herein may be used prophylactically in order to prevent infection lung inflammatory disease, including, for example pneumonia, bronchiolitis and/or bronchitis.

Exemplary carbohydrate binding modules (CBMs) may comprise the sialic acid binding domain of Vibrio cholerae NanH sialidase (VcCBM: a CBM40) and/or the equivalent (or homologous) domain from Streptococcus pneumoniae NanA sialidase (SpCBM: also a CBM40). Of course, similar or homologous sialic acid binding modules present in other organisms are to be encompassed within the scope of the term “CBM”.

An exemplary Vibrio cholerae NanH sialidase amino acid sequence is deposited under accession umber A5F7A4 and is reproduced below as SEQ ID NO: 1 (781 amino acids).

MRFKNVKKTA LMLAMFGMAT SSNAALFDYN
ATGDTEFDSP AKQGWMQDNT NNGSGVLTNA
DGMPAWLVQG IGGRAQWTYS LSTNQHAQAS
SFGWRMTTEM KVLSGGMITN YYANGTQRVL
PIISLDSSGN LVVEFEGQTG RTVLATGTAA
TEYHKFELVF LPGSNPSASF YFDGKLIRDN
IQPTASKQNM IVWGNGSSNT DGVAAYRDIK
FEIQGDVIFR GPDRIPSIVA SSVTPGVVTA
FAEKRVGGGD PGALSNTNDI ITRTSRDGGI
TWDTELNLTE QINVSDEFDF SDPRPIYDPS
SNTVLVSYAR WPTDAAQNGD RIKPWMPNGI
FYSVYDVASG NWQAPIDVTD QVKERSFQIA
GWGGSELYRR NTSLNSQQDW QSNAKIRIVD
GAANQIQVAD GSRKYVVTLS IDESGGLVAN
LNGVSAPIIL QSEHAKVHSF HDYELQYSAL
NHTTTLFVDG QQITTWAGEV SQENNIQFGN
ADAQIDGRLH VQKIVLTQQG HNLVEFDAFY
LAQQTPEVEK DLEKLGWTKI KTGNTMSLYG
NASVNPGPGH GITLTRQQNI SGSQNGRLIY
PAIVLDRFFL NVMSIYSDDG GSNWQTGSTL
PIPFRWKSSS ILETLEPSEA DMVELQNGDL
LLTARLDFNQ IVNGVNYSPR QQFLSKDGGI
TWSLLEANNA NVFSNISTGT VDASITRFEQ
SDGSHFLLFT NPQGNPAGTN GRQNLGLWFS
FDEGVTWKGP IQLVNGASAY SDIYQLDSEN
AIVIVETDNS NMRILRMPIT LLKQKLTLSQ
N

The CBM region of SEQ ID NO: 1 is from amino acid residue 25 to 216—this sequence may be SEQ ID NO: 2.

An exemplary Streptococcus pneumoniae NanA sialidase amino acid sequence has been deposited under accession number P62575 and is reproduced below as SEQ ID NO: 3 (1035 amino acids).

MSYFRNRDID IERNSMNRSV QERKCRYSIR
KLSVGAVSMI VGAVVFGTSP VLAQEGASEQ
PLANETQLSG ESSTLTDTEK SQPSSETELS
GNKQEQERKD KQEEKIPRDY YARDLENVET
VIEKEDVETN ASNGQRVDLS SELDKLKKLE
NATVHMEFKP DAKAPAFYNL FSVSSATKKD
EYFTMAVYNN TATLEGRGSD GKQFYNNYND
APLKVKPGQW NSVTFTVEKP TAELPKGRVR
LYVNGVLSRT SLRSGNFIKD MPDVTHVQIG
ATKRANNTVW GSNLQIRNLT VYNRALTPEE
VQKRSQLFKR SDLEKKLPEG AALTEKTDIF
ESGRNGKPNK DGIKSYRIPA LLKTDKGTLI
AGADERRLHS SDWGDIGMVI RRSEDNGKTW
GDRVTITNLR DNPKASDPSI GSPVNIDMVL
VQDPETKRIF SIYDMFPEGK GIFGMSSQKE
EAYKKIDGKT YQILYREGEK GAYTIRENGT
VYTPDGKATD YRVVVDPVKP AYSDKGDLYK
GNQLLGNIYF TTNKTSPFRI AKDSYLWMSY
SDDDGKTWSA PQDITPMVKA DWMKFLGVGP
GTGIVLRNGP HKGRILIPVY TTNNVSHLNG
SQSSRIIYSD DHGKTWHAGE AVNDNRQVDG
QKIHSSTMNN RRAQNTESTV VQLNNGDVKL
FMRGLTGDLQ VATSKDGGVT WEKDIKRYPQ
VKDVYVQMSA IHTMHEGKEY IILSNAGGPK
RENGMVHLAR VEENGELTWL KHNPIQKGEF
AYNSLQELGN GEYGILYEHT EKGQNAYTLS
FRKFNWDFLS KDLISPTEAK VKRTREMGKG
VIGLEFDSEV LVNKAPTLQL ANGKTARFMT
QYDTKTLLFT VDSEDMGQKV TGLAEGAIES
MHNLPVSVAG TKLSNGMNGS EAAVHEVPEY
TGPLGTSGEE PAPTVEKPEY TGPLGTSGEE
PAPTVEKPEY TGPLGTAGEE AAPTVEKPEF
TGGVNGTEPA VHEIAEYKGS DSLVTLTTKE
DYTYKAPLAQ QALPETGNKE SDLLASLGLT
AFFLGLFTLG KKREQ

The CBM region of SEQ ID NO: 3 is from amino acid residue 121 to 305—this sequence may be SEQ ID NO: 4.

Thus, CBMs for use as sialic acid binding molecules in the various aspects and embodiments of this disclosure may comprise a protein or peptide having the sequence of SEQ ID NO: 1, 2, 3 or 4 or a sequence or fragment derived therefrom. Sequences or fragments derived from any of SEQ ID NOS: 1, 2, 3 or 4 may themselves provide or encode a molecule with an ability to bind sialic acid (in other words a sialic acid binding molecule encoding portion of fragment of SEQ ID NOS: 1, 2, 3 or 4).

A sialic acid binding molecule for use may comprise an amino acid sequence which is at least 30%, at least 35%, at least 40%, at least 45%, at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to any of the sequences provided by SEQ ID NOS: 1, 2, 3 or 4.

Further, a sialic acid binding molecule for use may comprise from about residue 1, 5, 10, 15, 25 or 30 (i.e. from 1-30 or from any amino acid residue there between) to about residue 150, 175, 200, 210, 216, 220-781 (to any residue from 150 to 781 including any residue therebetween) of the V. cholerae sialidase molecule of SEQ ID NOS: 1 and 2. For example a sialic acid binding molecule for use may comprise a peptide having a sequence corresponding to residue 25 to about residue 216 of SEQ ID NO: 1 above.

A sialic acid binding molecule for use may comprise from about residue 80, 90, 100, 110, 120, 121 to 130 (i.e. from any of about residues 80 to 130 including any residue therebetween) to about residue 250, 275, 300, 305, 310, 320-1035 (i.e. to any residue from about 250-1035 including to about any residue therebetween) of the S. pneumoniae sialidase molecule of SEQ ID NOS: 3 and 4. For example, a sialic acid binding molecule for use may comprise a peptide having a sequence corresponding to residue 121 to about residue 305 of SEQ ID NO: 3 above.

A sialic acid binding molecule for use may comprise one or more CBMs. For example, suitable sialic acid binding molecules may comprise single CBM—for example a single sialic acid binding molecule derived from VcCBM (an exemplary VcCBM sequence being disclosed above as SEQ ID NOS: 1 and 2) or a single sialic acid binding molecule derived from SpCBM (an exemplary SpCBM sequence being disclosed above as SEQ ID NOS: 3 and 4). Alternatively, a sialic acid binding molecule for use may comprise a plurality or multiple (i.e. two or more) CBMs. Sialic acid binding molecules which comprise a plurality of CBMs may be termed “multivalent sialic acid binding molecules” or “multivalent CBMs”. A multivalent CBM may, for example, comprise two or more (for example three, four, five or six) sialic acid binding molecules derived from VcCBM or two or more sialic acid binding molecules derived from SpCBM. A multivalent CBM may comprise a mixture of different CBMs, for example one or more sialic acid binding molecules derived from VcCBMs with one or more sialic acid binding molecules derived from SpCBMs.

The sialic acid binding molecules for use may further comprise an oligomerisation domain. Suitable oligomerisation domains may exhibit an ability to self-associate to form multimer structures, for example trimers. An oligomerisation domain for use may comprise any molecule with the above mentioned oligomerisation properties or any functional fragment thereof. For example, one or more (for example two) sialic acid binding molecules (for example sialic acid binding molecules derived from the CBMs described herein) may be bound, coupled or fused to an oligomerisation domain—the resulting sialic acid binding molecule::oligomerisation domain “fusion” may then be used (with one or more other such “fusions”) as a molecule for modulating cell growth and/or activity and/or for treating or preventing any of the diseases and/or conditions disclosed herein.

Suitable oligomerisation domains may be derived from, for example, Pseudomonas aeruginosa pseudaminidase. An exemplary Pseudomonas aeruginosa pseudaminidase sequence amino acid sequence has been deposited under accession number PA0579 and is reproduced below as SEQ ID NO: 5 (438 amino acids).

MNTYFDIPHR LVGKALYESY YDHFGQMDIL
SDGSLYLIYR RATEHVGGSD GRVVFSKLEG
GIWSAPTIVA QAGGQDFRDV AGGTMPSGRI
VAASTVYETG EVKVYVSDDS GVTWVHKFTL
ARGGADYNFA HGKSFQVGAR YVIPLYAATG
VNYELKWLES SDGGETWGEG STIYSGNTPY
NETSYLPVGD GVILAVARVG SGAGGALRQF
ISLDDGGTWT DQGNVTAQNG DSTDILVAPS
LSYIYSEGGT PHVVLLYTNR TTHFCYYRTI
LLAKAVAGSS GWTERVPVYS APAASGYTSQ
VVLGGRRILG NLFRETSSTT SGAYQFEVYL
GGVPDFESDW FSVSSNSLYT LSHGLQRSPR
RVVVEFARSS SPSTWNIVMP SYFNDGGHKG
SGAQVEVGSL NIRLGTGAAV WGTGYFGGID
NSATTRFATG YYRVRAWI

The oligomerisation domain of SEQ ID NO: 5 is from amino acid residue 333 to 438—this sequence may be SEQ ID NO: 6.

Thus an oligomerisation domain for use may comprise an amino acid sequence which is at least 30%, at least 35%, at least 40%, at least 45%, at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to the sequences provided by SEQ ID NOS: 5 or 6.

Further, an oligomerisation domain for use may comprise from about residue 250, 275, 300, 310, 320, 333, 340 to 350 (i.e. from about residue 250 to about residue 350 including from about any residue therebetween) to about residue 400, 410, 420, 430 or 438 (i.e. to about any residue from about residue 400 residue 438 including to about any residue therebetween) of the P. aeruginosa pseudaminidase trimerisation domain (PaTD) provided by SEQ ID NO: 5. For example, a useful sialic acid binding molecule may exploit an oligomerisation domain comprising residues 333 to 438 of SEQ ID NO: 6.

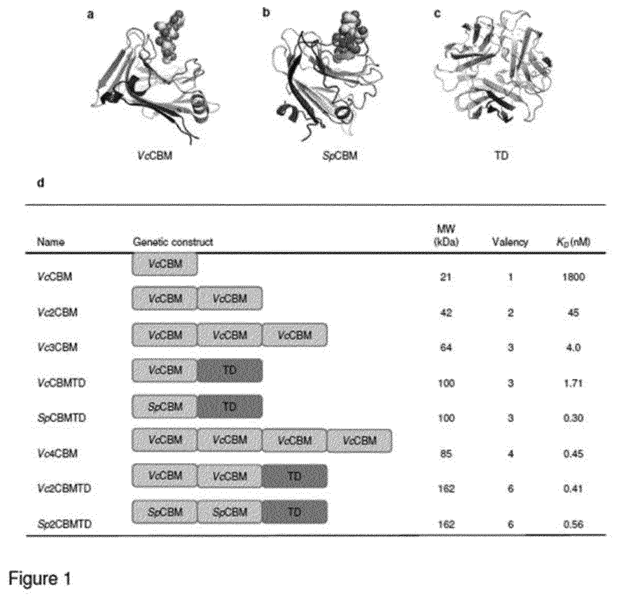

FIG. 1 describes a series of sialic acid binding molecules including molecules comprising (consisting essentially of, or consisting of) two or more VcCBMs optionally fused, bound or conjugated to an oligomerisation domain (such as a PaTD or oligomerisation fragment thereof). The sialic acid binding molecule may comprise, consist or consist essentially of two fused (or bound) VcCBMs which are, in turn, fused to an oligomerisation domain (see, for example, molecule Vc2CBMTD shown in FIG. 1).

Other sialic acid binding domains may comprise two or more SpCBMs optionally fused, bound or conjugated to an oligomerisation domain (such as a PaTD or an oligomerisation fragment thereof). Sialic acid binding molecules may comprise, consist or consist essentially of two fused (or bound) SpCBMs which are in turn fused to an oligomerisation domain (see, for example, molecule Sp2CBMTD shown in FIG. 1).

This disclosure refers to useful sialic acid binding molecules which are “derived” from the various sialic acid binding molecules described herein (including, for example, the various CBMs of this disclosure”. These “derived” (and useful) molecules may comprise sialic acid binding molecules which represent modified forms of any of the molecules described herein, including modified forms of the disclosed CBM sequences.

It should be understood that the term “modified” embraces molecules which contain one or more mutations relative to a reference sequence.

In the context of this disclosure, a “reference sequence” may be any wild type sequence encoding or providing a sialic acid binding molecule, for example a wild type CBM sequence. For example, a reference sequence may comprise, consist essentially of or consist of a wild type family 40 CBM sequence, e.g. the wild type CBM sequences from Vibrio cholerae NanH sialidase or Streptococcus pneumoniae NanA sialidase (it should be appreciated that similar or homologous CBMs (including CBM40s) present in other organisms are to be encompassed within the scope of the term “CBM” and/or as CBM reference sequences). A reference sequence from which a useful sialic acid binding molecule may be derived (including useful multivalent CBMs as described herein) may comprise any of the specific sequences described herein (for example SEQ ID NO: 1, 2, 3, 4 and 5.

For example, a modified CBM sequence for use may be derived from a specific or particular wild type CBM. A useful modified CBM sequence may comprise a wild type CBM sequence which includes one or more mutations.

The one or more mutation(s) may be functional. The mutations may, for example, alter the overall primary sequence of a CBM for use, but may not (substantially) alter the properties of the CBM—thus, while the sequence of a modified CBM may be different from the wild-type sequence from which it is derived, the overall function of the modified CBM is (substantially) identical to that of the wild-type CBM. Alternatively, the one or more mutation(s) may individually (and/or independently) or collectively (for example synergistically) modulate (improve or suppress/inhibit) one or more of the physiological, biological immunological and/or pharmacological properties characteristic of a wild type CBM (for example the wild type CBM from which the modified CBM is derived). In particular, the one or more mutations may:
(i) alter the immunogenicity (or antigenicity) of the CBM; and/or
(ii) alter (for example improve) the efficacy (of the CBM or of any multimeric molecule comprising a modified CBM)′ and/or
(iii) they may modulate (for example improve) the thermostability of the CBM; and/or
(iv) they may modulate (for example improve) the solubility of the CBM; and/or
(v) they may modulate (for example improve) the in vivo half-life of the molecule.

A “mutation” may include any alteration to a wild-type CBM molecule. For example, the term “mutation” may embrace, for example:
(i) one or more amino acid substitution(s) (where one or more of the wild type amino acid(s) is/are swapped or changed for another (different) amino acid—the term “substitutions” would include conservative amino acid substitutions); and/or
(ii) one or more amino acid deletion(s) (where one or more of the wild type amino acid residue(s) are removed); and/or
(iii) one or more amino acid addition(s)/insertion(s) (where additional amino acid residue(s) are added to a wild type (or reference) primary sequence); and/or
(iv) one or more amino acid/sequence inversions (usually where two or more consecutive amino acids in a primary sequence are reversed; and/or
(v) one or more amino acid/sequence duplications (where an amino acid or a part of the primary amino acid sequence (for example a stretch of 5-10 amino acids) is repeated)

Thus, a useful modified CBM (i.e. a CBM for use in the medical uses and methods described herein) may comprise one or more of the mutations described herein.

By way of non-limiting example, the following represent individual units (referred to as “HEX” units) which may be used to make hexameric sialic acid binding molecules which have a particular application in the various compositions, medicaments, methods and uses described herein (for example for use in methods of treating or preventing lung inflammatory diseases, including pneumonia and/or bronchitis). In each case, the HEX unit comprises two modified CBMs (denoted CBM1 and CBM2), which modified CBMs are derived from the sialic acid binding domain of Streptococcus pneumoniae NanA sialidase (SpCBM: a CBM40 family member). The specific mutations introduced to each modified CBM are identified in parenthesis. It should be noted that a “- - - -” symbol indicates an amino acid linker (linking one CBM to another or a CBM to an oligomerisation domain). As such, a hexameric (‘HEX’) sialic acid binding molecule may be made up of several (for example 3) HEX units. The oligomerisation domain (denoted “TD”) conjugates the units together as a trimer. While any given hexamer may comprise identical copies of the units described above (and below under the headings HEX1 unit, HEX2 unit, HEX3 unit, HEX4 unit, HEX5 unit, HEX6 unit and HEX17 unit), one of skill will appreciate that further options are available. For example, a HEX unit may be made up of two CBMs, each having different mutations (the mutations being one or more selected from the options detailed herein).

(i) HEX1 unit
CBM1(L170T V239A V246G I286A Y292E)-----CBM2(L170T V239A V246G I286A
Y292E)-----TD(S342D L348D R403K)
(ii) HEX2 unit
CBM1(V239A V246G I286A Y292E)-----CBM2(V239A V246G I286A Y292E)-----TD
(S342D R403K)
(iii) HEX3 unit
CBM1(V239A V246G I286A)-----CBM2(V239A V246G I286A)-----TD(S342D R403K)
(iv) HEX4 unit
CBM1(V239A V246G)-----CBM2(V239A V246G)-----TD(S342D)
(v) HEX5 unit
CBM1(V239A V246G)-----CBM2(V239A V246G)-----TD(R403K)
(vi) HEX6 unit
CBM1(V239A V246G)-----CBM2(V239A V246G)-----TD(S342D R403K)
(vii) HEX17unit
CBM1(V239A V246G A162P)-----CBM2(V239A V246G A162P)-----TD(S342D R403K)

It will be noted that HEX6 and HEX17 are identical except for the additional A162P mutation. This proline mutation (a substitution for the wild type Alanine at residue 162) has been shown to improve thermostability (the single CBM Tm by 3-4° C.). Further information regarding the use of proline mutations may be derived from Fu 2009, ‘Increasing protein stability by improving beta-turns’ (DOI 10.1002/prot.22509) which describes the general approach. The proline mutation does not affect (increase or decrease) the predicted immunogenicity of the CBM molecule, is not located near the other mutations, the N- or C-termini or the ligand binding site. Rather unexpectedly, beyond the modest improvement in thermostability, it was noted that the A162P mutation yields a hexameric CBM (i.e. a molecule comprising 3×HEX17 units) exhibiting a marked improvement in in vivo experiments—in particular in comparison to those same experiments conducted using a hexameric molecule comprising 3×Hex6 units. For example, the modified molecules (in particular a molecule comprising 3×HEX17 units) exhibit modulation over pro-inflammatory cytokines, including for example IL-8. Indeed the modulatory effect (specifically an inhibitory effect) on the production of IL-8 by a molecule comprising 3×HEX17 units, was improved over other tested modified molecules.

Relative to the amino acid sequences of Sp2CBMTD (aka “SpOrig” SEQ ID NO: 7) the amino acid sequence of the HEX6 (SEQ ID NO: 8) and HEX 17 (SEQ ID NO: 9) molecules is:

| | |
|---|---|
| SpOrig | GAMVIEKEDVETNASNGQRVDLSSELDKLKKLENATVHMEFKPDAKAPAFYNLFSVSSAT |
| HEX6 | GAMVIEKEDVETNASNGQRVDLSSELDKLKKLENATVHMEFKPDAKAPAFYNLFSVSSAT |
| Hex17 | GAMVIEKEDVETNASNGQRVDLSSELDKLKKLENATVHMEFKPDPKAPAFYNLFSVSSAT |
| SpOrig | KKDEYFTMAVYNNTATLEGRGSDGKQFYNNYNDAPLKVKPGQWNSVTFTVEKPTAELPKG |
| HEX6 | KKDEYFTMAVYNNTATLEGRGSDGKQFYNNYNDAPLKVKPGQWNSVTFTVEKPTAELPKG |
| Hex17 | KKDEYFTMAVYNNTATLEGRGSDGKQFYNNYNDAPLKVKPGQWNSVTFTVEKPTAELPKG |
| SpOrig | RVRLYVNGVLSRTSLRSGNFIKDMPDVTHVQIGATKRANNTVWGSNLQIRNLTVYNRALT |
| HEX6 | RARLYVNGGLSRTSLRSGNFIKDMPDVTHVQIGATKRANNTVWGSNLQIRNLTVYNRALT |
| Hex17 | RARLYVNGGLSRTSLRSGNFIKDMPDVTHVQIGATKRANNTVWGSNLQIRNLTVYNRALT |
| SpOrig | PEEVQKRSGGGSGVIEKEDVETNASNGQRVDLSSELDKLKKLENATVHMEFKPDAKAPAF |
| HEX6 | PEEVQKRSGGGSGVIEKEDVETNASNGQRVDLSSELDKLKKLENATVHMEFKPDAKAPAF |
| Hex17 | PEEVQKRSGGGSGVIEKEDVETNASNGQRVDLSSELDKLKKLENATVHMEFKPDPKAPAF |
| SpOrig | YNLFSVSSATKKDEYFTMAVYNNTATLEGRGSDGKQFYNNYNDAPLKVKPGQWNSVTFTV |
| HEX6 | YNLFSVSSATKKDEYFTMAVYNNTATLEGRGSDGKQFYNNYNDAPLKVKPGQWNSVTFTV |
| Hex17 | YNLFSVSSATKKDEYFTMAVYNNTATLEGRGSDGKQFYNNYNDAPLKVKPGQWNSVTFTV |
| SpOrig | EKPTAELPKGRVRLYVNGVLSRTSLRSGNFIKDMPDVTHVQIGATKRANNTVWGSNLQIR |
| HEX6 | EKPTAELPKGRARLYVNGGLSRTSLRSGNFIKDMPDVTHVQIGATKRANNTVWGSNLQIR |
| Hex17 | EKPTAELPKGRARLYVNGGLSRTSLRSGNFIKDMPDVTHVQIGATKRANNTVWGSNLQIR |
| SpOrig | NLTVYNRALTPEEVQKRSGGALGVPDFESDWFSVSSNSLYTLSHGLQRSPRRVVVEFARS |
| HEX6 | NLTVYNRALTPEEVQKRSGGSLGVPDFESDWFDVSSNSLYTLSHGLQRSPRRVVVEFARS |
| Hex17 | NLTVYNRALTPEEVQKRSGGSLGVPDFESDWFDVSSNSLYTLSHGLQRSPRRVVVEFARS |
| SpOrig | SSPSTWNIVMPSYFNDGGHKGSGAQVEVGSLNIRLGTGAAVWGTGYFGGIDNSATTRFAT |
| HEX6 | SSPSTWNIVMPSYFNDGGHKGSGAQVEVGSLNIKLGTGAAVWGTGYFGGIDNSATTRFAT |
| Hex17 | SSPSTWNIVMPSYFNDGGHKGSGAQVEVGSLNIKLGTGAAVWGTGYFGGIDNSATTRFAT |
| SpOrig | GYYRVRAWI |
| HEX6 | GYYRVRAWI |
| Hex17 | GYYRVRAWI |

The disclosed molecules, including those for use in the described compositions, medicaments and methods may be generated using PCR-based cloning techniques and a suitable method for the generation of multivalent molecules of this type is described in, for example, Connaris et al, 2009 (Enhancing the Receptor Affinity of the Sialic Acid-Binding Domain of Vibrio cholerae Sialidase through Multivalency; J. Biol. Chem; Vol. 284(11); pp 7339-7351). For example, multivalent CBM molecules, including the likes of HEX17 and/or HEX17 variants may be prepared as constructs comprising multiple sialic acid binding molecules (for example modified CBMs) linked by amino acid/peptide linkers.

As stated, each CBM (for example modified CBM) may be linked to another by, for example, peptides comprising 5, 10 or 15 amino acids. By way of example any one or more of the following peptides may be used to link two or more CBMs (including the described modified CBMs) to produce a multivalent CBM:

i) 5 amino acid linkers:
ALXGS
LQALG
GGXSG
GGALG
GGGGS
ii) 10 amino acid linkers:
ALXGSGGGSG
LQALGGGGSL
iii) 15 amino acid linkers:
ALXGSGGGSGGGGSG

where “X” is any amino acid.

Thus an exemplary sialic acid binding molecule (for example a HEX17 unit) may take the following form:

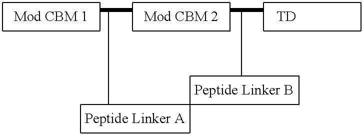

This schematic shall be referred to hereinafter as General Formula 1.

Thus, a HEX17 unit may conform to General Formula 1 as set out above, comprising two modified CBMs (“Mod CBM 1” and “Mod CBM 2”) and a trimerisation domain, TD wherein Peptide Linkers A and/or B are selected from the linker options presented above as (i), (ii) and/or (iii).

It should be noted that the term “HEX17” embraces not only the complete HEX17 sequence described above but also functional (for example, sialic acid binding and/or anti-inflammatory) fragments derived therefrom. Indeed, each of the “Mod CBM” units shown in General Formula 1 may be the HEX17 units shown above.

In view of the above, a HEX17 molecule for use in the various aspects of this disclosure comprises three HEX17 units bound together via the trimerisation domain.

Accordingly, the various uses, compositions, sialic acid binding molecules for use, methods and medicaments described herein may exploit sialic acid binding molecules which comprise, consist of or consist essentially of sialic acid binding molecules selected from the group consisting of:
(i) one or more modified CBM(s);
(ii) a functional HEX17 fragment;
(iii) a sialic acid binding molecule comprising a sequence which is at least 30%, at least 35%, at least 40%, at least 45%, at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to SEQ ID NO: 9; and
(iv) a HEX17 molecule (i.e. a sialic acid binding molecule comprising, consisting of or consisting essentially of SEQ ID NO: 9)

For the avoidance of doubt, the term “HEX17” includes sialic acid binding molecules having a sequence corresponding to that provided by SEQ ID NO: 9 or which exhibits some level of sequence identity thereto (for example, at least 30%, at least 35%, at least 40%, at least 45%, at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to SEQ ID NO: 9). Sialic acid molecules with sequences exhibiting some level of identity to SEQ ID NO: 9 (which level of identity is selected from the % identity values disclosed above) may be referred to as “HEX17 variants”. HEX17 variants may be functional in that they bind to sialic acid and/or are anti-inflammatory (i.e. they inhibit the production or expression of certain proinflammatory cytokines).

As such, the present disclosure provides:
HEX17 and/or a HEX17 variant;
for use in the treatment and/or prevention of a lung inflammatory disease, pneumonia, bronchiolitis and/or bronchitis.

The disclosure also provides the use of HEX17 and/or a HEX17 variant in the manufacture of a medicament for use in the treatment and/or prevention of a lung inflammatory disease, pneumonia, bronchiolitis and/or bronchitis.

The disclosure also relates to a method of treating or preventing a lung inflammatory disease, pneumonia, bronchiolitis and/or bronchitis, said method comprising the steps of administering to a subject in need thereof, a therapeutically effective amount of HEX17 and/or a HEX17 variant.

For completeness, it should be noted that Vc2CBM comprises, consists essentially of or consists of two Vibrio cholerae NanH sialidase CBM units linked, bound or conjugated together. An exemplary Vc2CBM sequence may comprise, consist essentially of or consist of:

GAMALEDYNATGDTEFDSPAKQGWMQDNTNNGSGVLTNADGMPAWLVQG
IGGRAQWTYSLSTNQHAQASSFGWRMTTEMKVLSGGMITNYYANGTQRV
LPIISLDSSGNLVVEFEGQTGRTVLATGTAATEYHKEELVFLPGSNPSA
SFYFDGKLIRDNIQPTASKQNMIVWGNGSSNTDGVAAYRDIKFEIQGDA
LNGSMALFDYNATGDTEFDSPAKQGWMQDNTNNGSGVLTNADGMPAWLV
QGIGGRAQWTYSLSTNQHAQASSFGWRMTTEMKVLSGGMITNYYANGTQ
RVLPIISLDSSGNLVVEFEGQTGRTVLATGTAATEYHKFELVFLPGSNP
SASFYFDGKLIRDNIQPTASKONMIVWGNGSSNTDGVAAYRDIKFEIQG
D

Additionally, Vc4CBM comprises, consists essentially of or consists of four Vibrio cholerae NanH sialidase CBM units linked, bound or conjugated together. An exemplary Vc4CBM sequence may comprise, consist essentially of or consist of the following sequence:

GAMALEDYNATGDTEFDSPAKQGWMQDNTNNGSGVLTNADGMPAWLVQGIGGRAQWTYSLSTNQHAQA
SSFGWRMTTEMKVLSGGMITNYYANGTQRVLPIISLDSSGNLVVEFEGQTGRTVLATGTAATEYHKFE
LVFLPGSNPSASFYFDGKLIRDNIQPTASKQNMIVWGNGSSNTDGVAAYRDIKFEIQGDALNGSMALF
DYNATGDTEFDSPAKQGWMQDNTNNGSGVLTNADGMPAWLVQGIGGRAQWTYSLSTNQHAQASSFGWR
MTTEMKVLSGGMITNYYANGTQRVLPIISLDSSGNLVVEFEGQTGRTVLATGTAATEYHKFELVFLPG
SNPSASFYFDGKLIRDNIQPTASKQNMIVWGNGSSNTDGVAAYRDIKFEIQGDLQALGMALFDYNAT
GDTEFDSPAKQGWMQDNTNNGSGVLTNADGMPAWLVQGIGGRAQWTYSLSTNQHAQASSFGWRMTTEM
KVLSGGMITNYYANGTQRVLPIISLDSSGNLVVEFEGQTGRTVLATGTAATEYHKFELVFLPGSNPSA
SFYFDGKLIRDNIQPTASKQNMIVWGNGSSNTDGVAAYRDIKFEIQGDGGNSGMALFDYNATGDTEFD
SPAKQGWMQDNTNNGSGVLTNADGMPAWLVQGIGGRAQWTYSLSTNQHAQASSFGWRMTTEMKVLSGG
MITNYYANGTQRVLPIISLDSSGNLVVEFEGQTGRTVLATGTAATEYHKFELVFLPGSNPSASFYFDG
KLIRDNIQPTASKONMIVWGNGSSNTDGVAAYRDIKFEIQGD

Also, Sp2CBM comprises, consists essentially of or consists of two Streptococcus pneumoniae NanA sialidase units linked, bound or conjugated together. An exemplary Sp2CBM sequence may comprise, consist essentially of, or consist of two copies of the following sequence:

GSMVIEKEDVETNASNGQRVDLSSELDKLKKLENATVHMEFKPDAKAPA
FYNLFSVSSATKKDEYETMAVYNNTATLEGRGSDGKQFYNNYNDAPLKV
KPGQWNSVTFTVEKPTAELPKGRVRLYVNGVLSRTSLRSGNFIKDMPDV
THVQIGATKRANNTVWGSNLQIRNLTVYNRALTPEEVQKRS

The two copies of the abovementioned sequence may be joined via any one of the peptide linker sequences described herein. For example, a Sp2CBM sequence may comprise, consist essentially of, or consist of

GSMVIEKEDVETNASNGQRVDLSSELDKLKKLENATVHMEFKPDAKAPAFYNLFSVSSATKKDEYETM
AVYNNTATLEGRGSDGKQFYNNYNDAPLKVKPGQWNSVTFTVEKPTAELPKGRVRLYVNGVLSRTSLR
SGNFIKDMPDVTHVQIGATKRANNTVWGSNLQIRNLTVYNRALTPEEVQKRS[xxxxx][xxxxxxxx
xx][xxxxxxxxxxxxxxx]GSMVIEKEDVETNASNGQRVDLSSELDKLKKLENATVHMEFKPDAKAP
AFYNLFSVSSATKKDEYFTMAVYNNTATLEGRGSDGKQFYNNYNDAPLKVKPGQWNSVTFTVEKPTAE
LPKGRVRLYVNGVLSRTSLRSGNFIKDMPDVTHVQIGATKRANNTVWGSNLQIRNLTVYNRALTPEEV
QKRS

Wherein [xxxxx], [xxxxxxxxxx] and [xxxxxxxxxxxxxxx]—represent the choice of linker peptide sequences as outlined below. Both CBM sequences would be joined by one of the 5, 10 or 15 amino acid linker sequences described herein.

Vc2CBM and Vc4CBM may be described as tandem-repeat multivalent proteins based on the Family 40 sialic acid binding domain (CBM) of the nanH gene encoding the sialidase from V. cholerae. Sp2CBM may be described as a tandem-repeat multivalent protein based on the family 40 sialic acid binding domain (CBM) of the nanA gene encoding the sialidase from S. pneumoniae.

Further, it should be noted that the various uses, methods and medicaments described herein may exploit one or more of the sialic acid binding molecules described herein. For example, a sialic acid binding molecule comprising HEX17 (i.e. a sequence according to SEQ ID NO: 9) or a HEX17 variant may be administered to a subject together, concurrently or separately with another sialic acid binding molecule or and/or other therapeutic moiety or adjuvant.

The present disclosure provides compositions for the various uses, medicaments compositions, and methods described herein. As such, any of the useful sialic acid binding molecule(s) (for example the HEX17 or HEX17 variants) described herein may be formulated for subsequent use. For example, a sialic acid binding molecule (or molecules) may be formulated as therapeutic or pharmaceutical compositions. The various compositions may comprise one or more of the sialic acid binding molecules described herein and any given treatment may require the administration (together, concurrently or separately) of one or more of these compositions.

Pharmaceutical compositions according to the present invention, in particular those formulations for mucosal or intranasal administration may be prepared conventionally, comprising substances that are customarily used in pharmaceuticals and as described in, for example, Remington's The Sciences and Practice of Pharmacy, 22nd Edition (Pharmaceutical Press 2012) and/or Handbook of Pharmaceutical Excipients, 7th edition (compiled by Rowe et al, Pharmaceutical Press, 2012)—the entire content of all of these documents and references being incorporated by reference.

Any suitable amount of a sialic acid binding molecule (for example, the HEX17 molecules described herein) may be used. For example, whether a composition comprising a sialic acid binding molecule (for example HEX17) is to be administered intravenously or mucosally (for example, intranasally) the dose of sialic acid binding molecule may comprise anywhere between about 0.1 μg and about 1000 μg. For example, a dose of about (for example +/−0.5 μg) 0.1 μg, 0.5 μg, 1 μg, 5 μg, 10 μg, 11 μg, 12 μg, 13 μg, 14 μg, 15 μg, 20 μg, 30 μg, 40 μg, 50 μg, 100 μg, 200 μg, 300 μg, 400 μg, 500 μg, 600 μg, 700 μg, 800 μg, 900 μg or 950 μg of the sialic acid binding molecule may be used. These amounts may be provided in any suitable volume of excipient, diluent or buffer. For example, the amount of sialic acid binding molecule may be provided in anywhere between about 1 μl to about 5 ml of excipient, diluent or buffer. For example, the required amount of sialic acid binding molecule may be combined (or formulated) with about 5 μl, 10 μl, 15 μl, 20 μl, 25 μl, 30 μl, 35 μl, 40 μl, 45 μl, 50 μl, 55 μl, 60 μl, 65 μl, 70 μl, 75 μl, 80 μl, 85 μl, 90 μl, 95 μl, 100 μl, 200 μl, 300 μl, 400 μl, 500 μl, 600 μl, 700 μl, 800 μl, 900 μl, 1 ml, 2 ml, 3 ml or 4 ml. Concentrations of 0.1-1 mg (sialic acid binding protein) per ml (excipient, diluent or buffer) may be most useful.

A composition of this disclosure (for example, a composition comprising HEX17 or a HEX17 variant) may be administered (prophylactically) to a subject at regular and/or predetermined times. For example, a composition described herein may be administered to at regular and/or predetermined times both before, during and after the subject enters or encouters a scenario during which they might be vulnerable and/or susceptible to a lung inflammatory disease (such as, for example pneumonia and/or bronchitis). A composition of this disclosure may be administered every day and/or every few days. A composition of this disclosure may be administered multiple times throughout any given day. A composition described herein may be administered over a period of weeks or months or years. The precise administration regimen will depend on the subject, the health of that subject and the period of time that subject is deemed to be at risk of or vulnerable to, a lung inflammatory disease (such as pneumonia or bronchitis).

### DETAILED DESCRIPTION

The present invention will now be described in detail with reference to the following figures which show:

FIG. 1: Building blocks of the multivalent CBM forms and their affinities for sialic acid. a, VcCBM, residues 25-216 of the V. cholerae sialidase (PDB:1w0p) with α-2,3-sialyllactose drawn as spheres. b, SpCBM, residues 121-305 of S. pneumoniae NanA sialidase with α-2,3-sialyllactose (PDB:4c1w). c, TD, the trimerisation domain, residues 333-438, of the P. aeruginosa pseudaminidase (PDB:2w38) in rainbow colours; the other two monomers in single colors. d, Multivalent forms: their molecular weights, valencies and binding affinities for α2,3-sialyllactose as determined by surface plasmon resonance (SPR) at 25° C. (K_{D} values for VcCBM, Vc2CBM and Vc3CBM had been reported previously⁷). Tandem repeat CBMs, and oligomeric CBMs fused to TD are linked by a 5-amino linker.

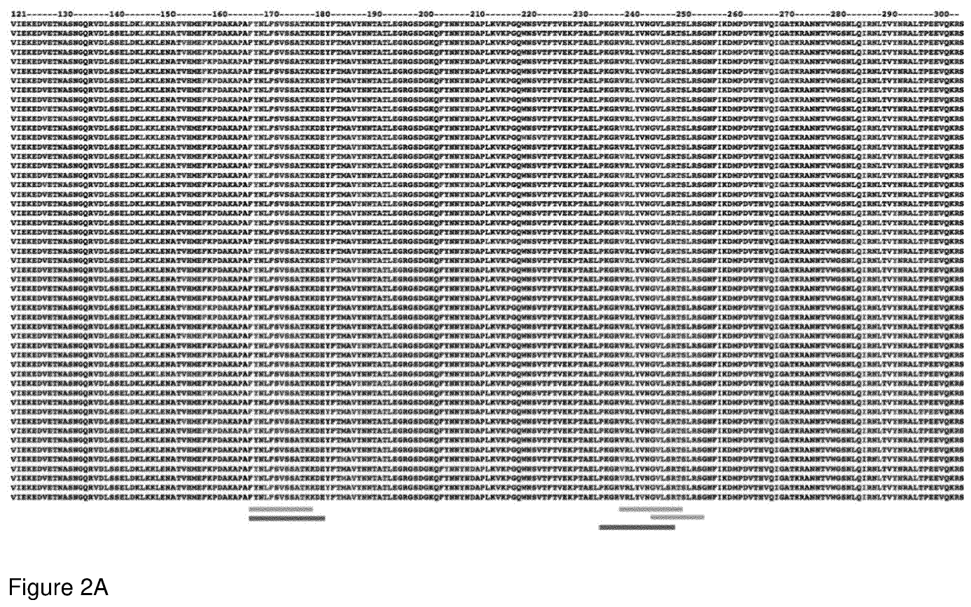

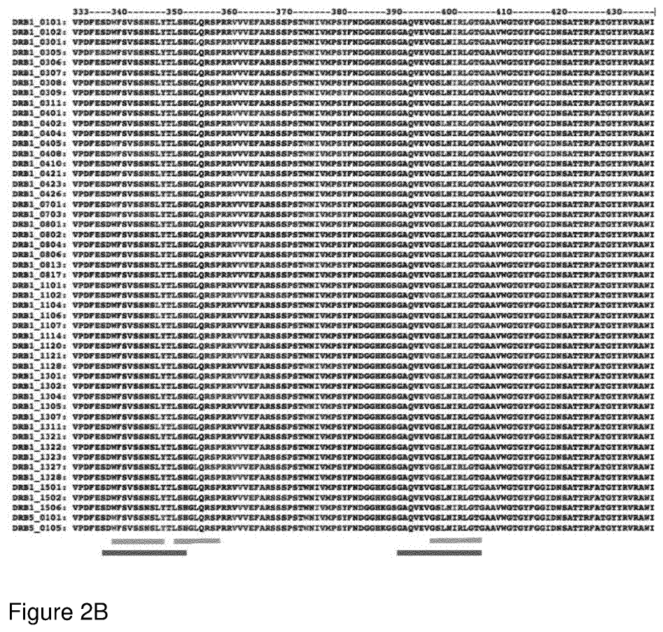

FIG. 2: ProPred predictions of antigenic peptides. A. SpCBM sequence. B. PaTD sequence. Predicted binders are coloured blue, with the first residue of each binding region shown in red. Antigenic peptides predicted by Nordic Biopharma (green bars) and ProImmune (purple bars) are shown under the sequences.

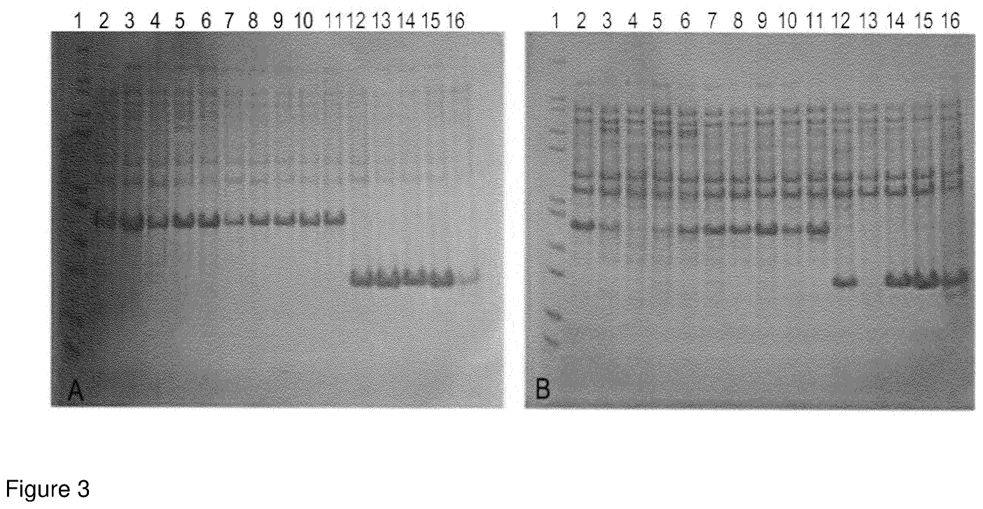

FIG. 3: Expression test of wild type and mutated domains. Lane 1, M12 standard; Lane 2, WTSpCBM; Lanes 3-11, Im15-Im23; Lanes 12-15, Im24-Im27; Lane 16, WT PaTD. A) Whole cell extracts, B) Soluble extracts.

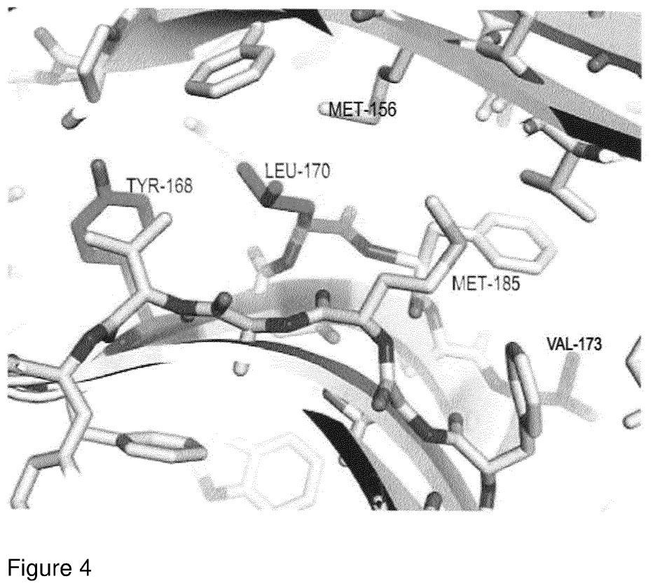

FIG. 4: Position of peptide 167-181 in the SpCBM structure

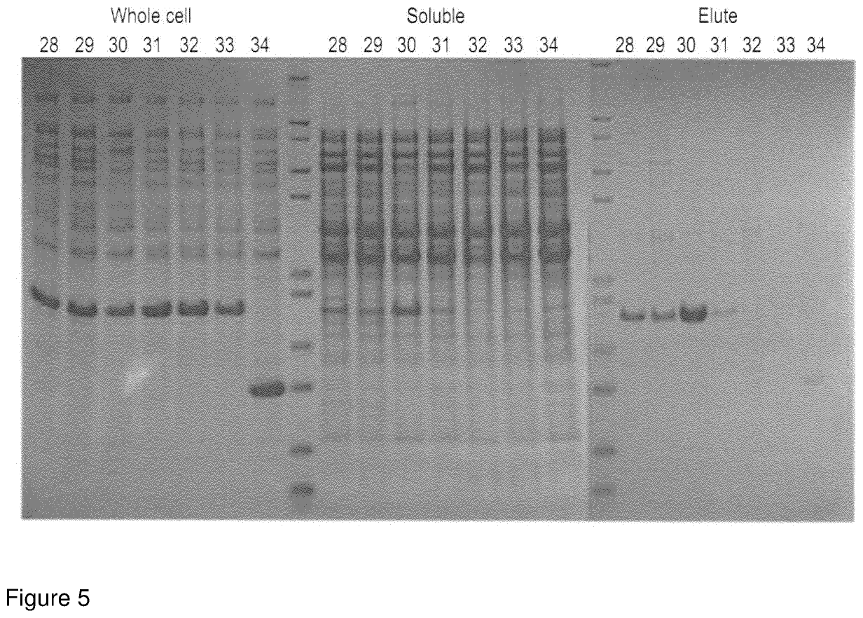

FIG. 5: Expression and Ni-NTA pull-down of variants Im28 to Im34

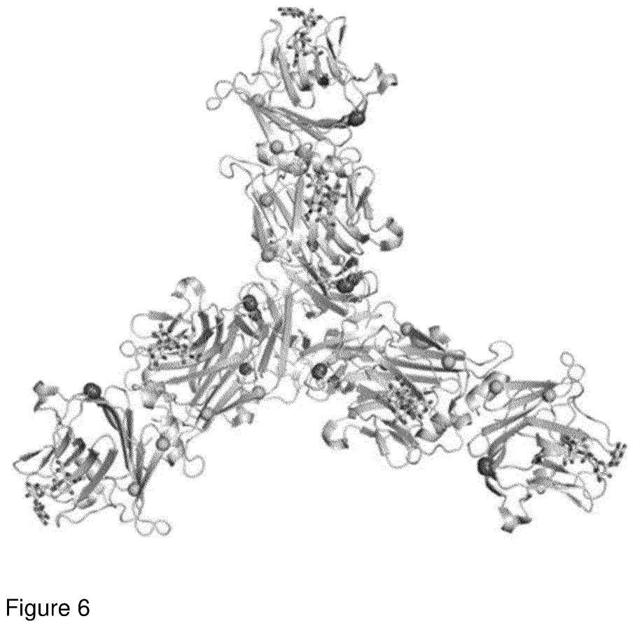

FIG. 6: Sites of the HEX17 mutations on the hexamer structure. The quarternary structure of HEX17 was modelled by assembling the crystal structures of the individual SpCBM (pdb code 4c1x) and PaTD (pdb code 2w38) into the hexamer (i.e. 6 copies of SpCBM and 3 copies of PaTD per molecule). The positions of the bound ligand (α2,3-sialyllactose) are shown in stick form (orange). The positions of the mutations are also shown: Blue, the sites of the A162P mutation; Cyan, the sites of the other two CBM mutations; Magenta, the sites of the TD mutations.

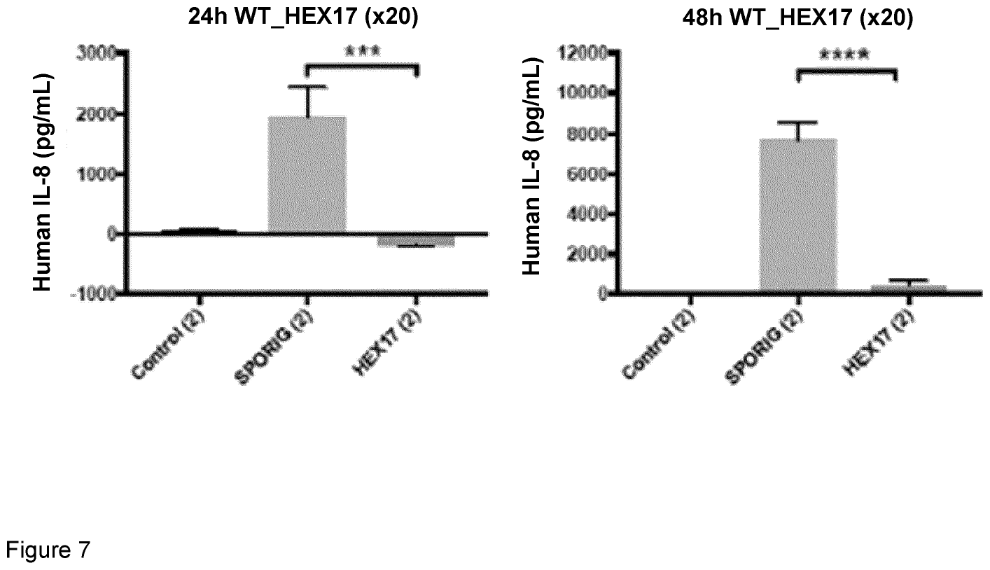

FIG. 7: IL-8 stimulation. A549 cells were stimulated by the addition of 10 μg of biologic (Hex17). Cell supernatant was harvested at 24 or 48 h time-points and the IL-8 content was determined by ELISA. Statistical significance between control and treated cells was determined with one-way ANOVA using Tukey's multiple comparison test.

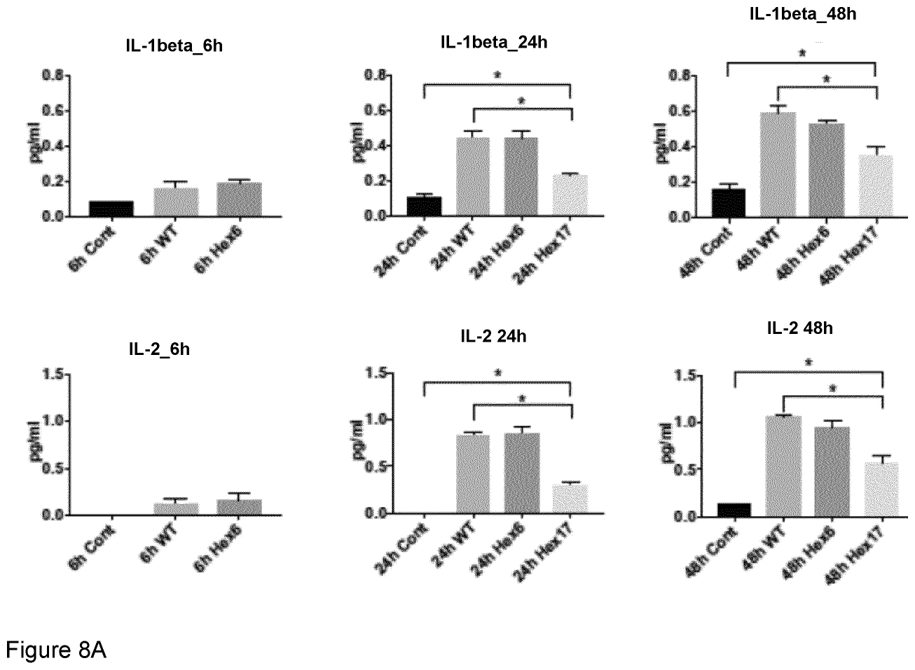

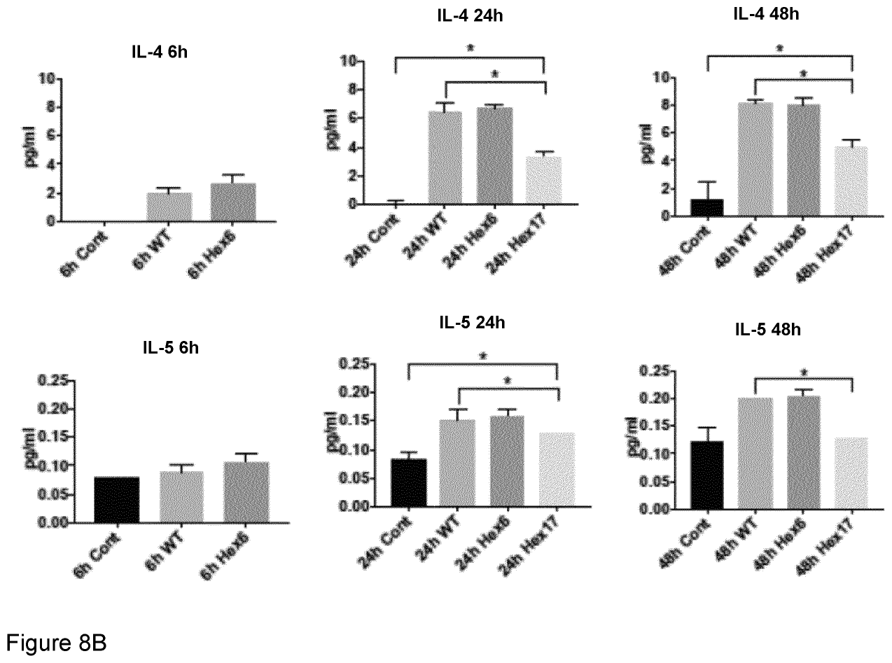

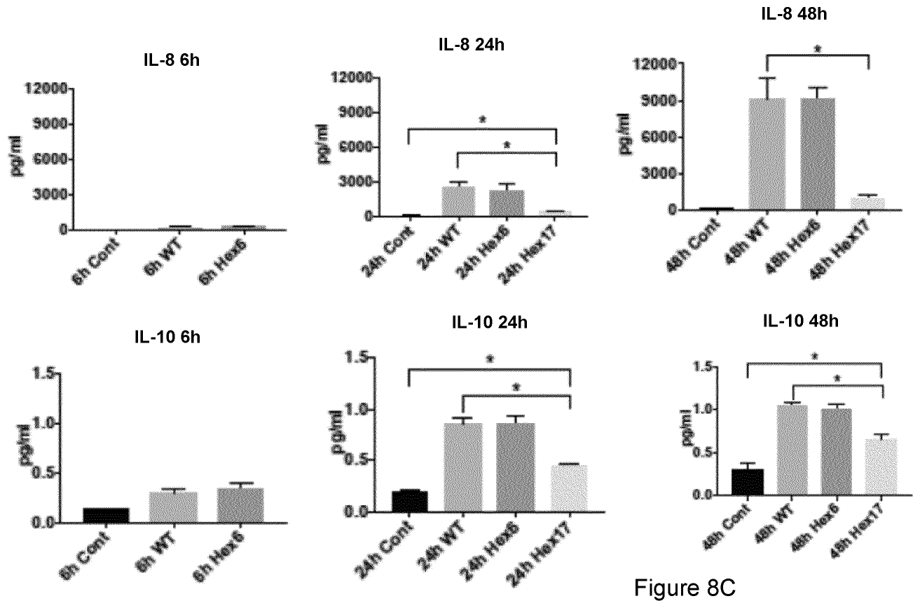

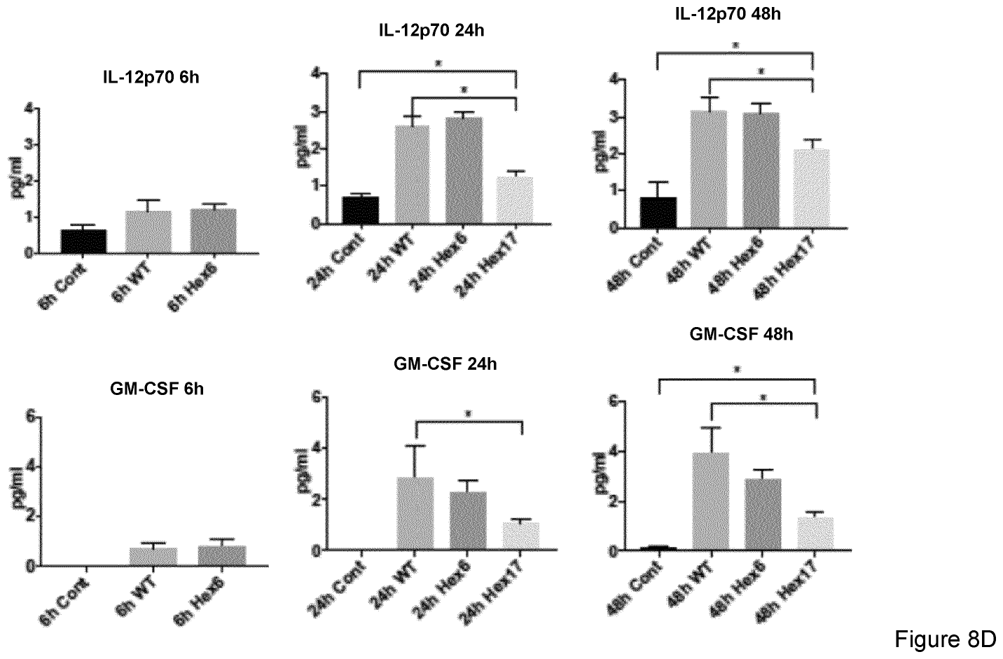

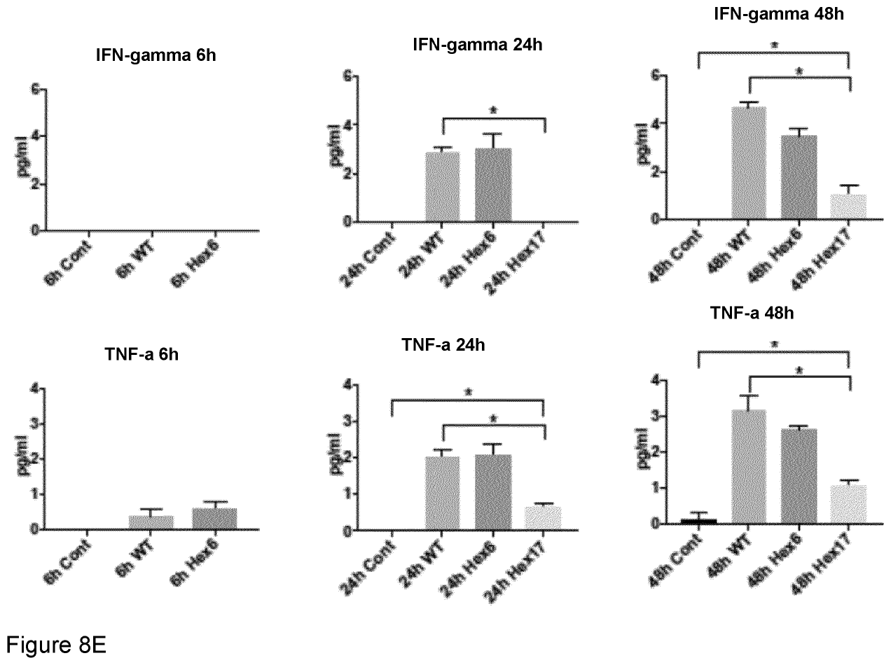

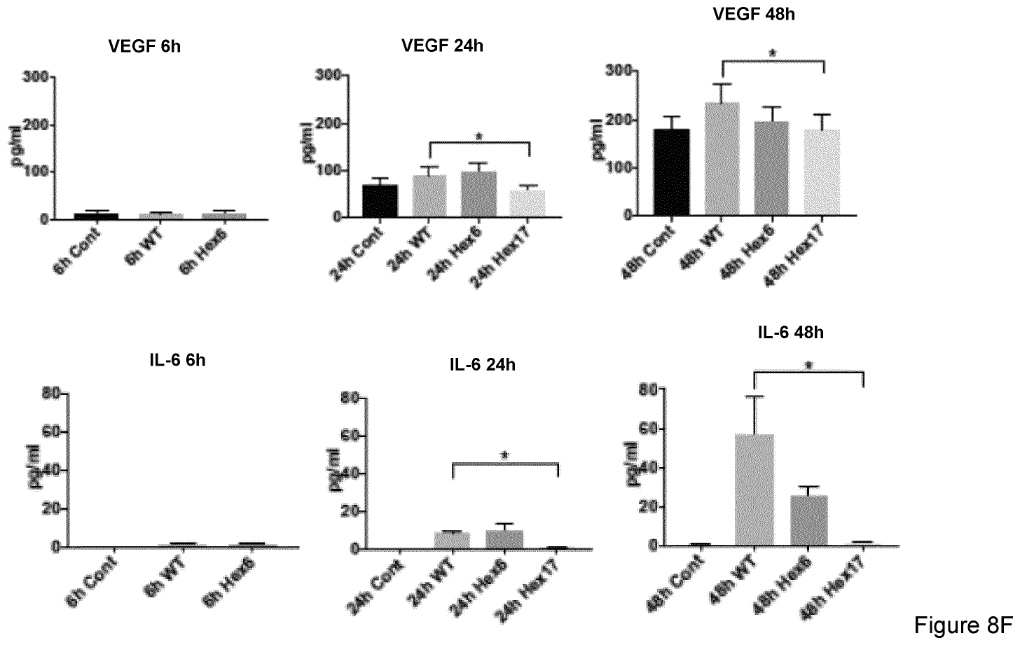

FIG. 8: Multiplex analysis of inflammatory mediators. A549 cells were stimulated by the addition of 10 μg of biologic (Sp2CBMTD (aka SpOrig), HEX6 or HEX17). Cell supernatant was harvested at 6 h, 24 or 48 h time-points and inflammatory mediators analysed using a Human Cytokine 12-plex Assay. Statistical significance between control and/or WT hexamer and hexamer variants was determined using a one-way ANOVA (Tukey's multiple comparison test).

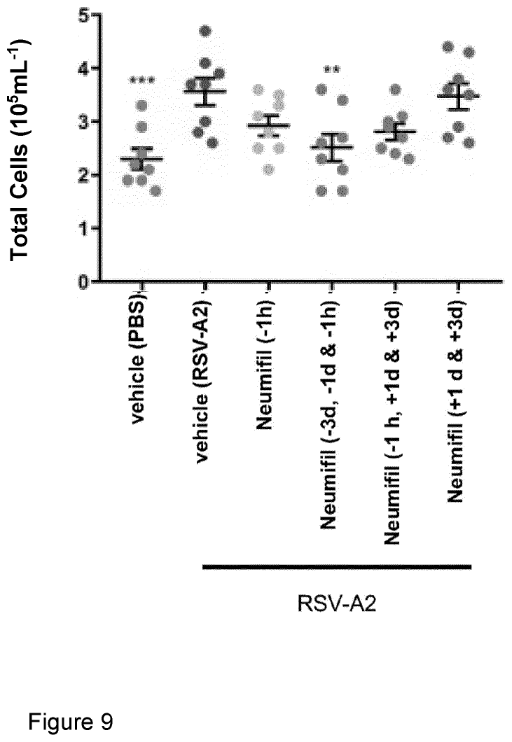

FIG. 9: Scatter plot showing the effect on total number of cells in bronchoalveolar lavage fluid (BALF) of mice infected with RSV-A2 and treated with Neumifil (HEX17: 100 μg, i.n.). Groups represent vehicle (PBS) control (group 1), vehicle (RSV-A2) control (group 2), and Neumifil-treated groups dosed 1 hour before infection (group 3); 3 days, 1 day and 1 hour before infection (group 4); 1 hour before infection, 1 day and 3 days after infection (group 5); 1 day and 3 days post infection (group 6). Each symbol represents total cells for each individual animal per group and each line. Each column represents the group mean and each bar represents standard error of mean (SEM) of n=8 animals. Changes in each treatment group were compared to RSV-infected animals treated with vehicle using Dunnett's one-way analysis of variance. ** P<0.01, *** P<0.001.

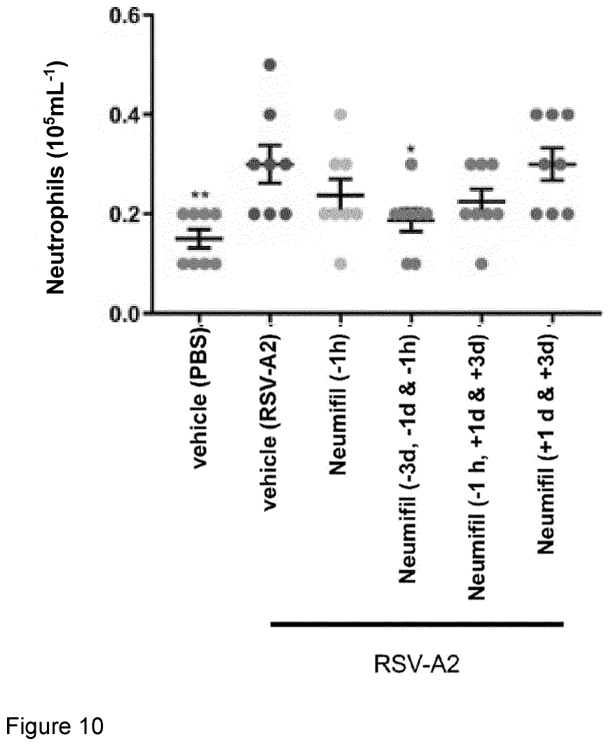

FIG. 10. Scatter plot showing the effect on neutrophil numbers in bronchoalveolar lavage fluid (BALF) mice infected with RSV-A2 and treated with Neumifil (HEX17: 100 μg, i.n). Groups represent vehicle (PBS) control (group 1), vehicle (RSV-A2) control (group 2), and Neumifil-treated groups dosed 1 hour before infection (group 3); 3 days, 1 day and 1 hour before infection (group 4); 1 hour before infection, 1 day and 3 days after infection (group 5); 1 day and 3 days post infection (group 6). Each symbol represents total cells for each individual animal per group and each line. Each column represents the group mean and each bar represents standard error of mean (SEM) of n=8 animals. Changes in each treatment group were compared to RSV-infected animals treated with vehicle using Dunnett's one-way analysis of variance. * P<0.05, ** P<0.01.

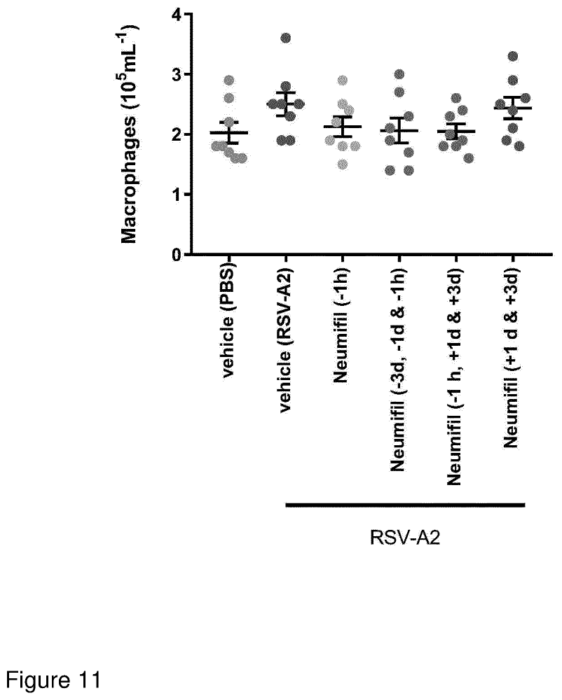

FIG. 11. Scatter plot showing the effect on macrophage numbers in bronchoalveolar lavage fluid (BALF) mice infected with RSV-A2 and treated with Neumifil (100 μg, i.n). Groups represent vehicle (PBS) control (group 1), vehicle (RSV-A2) control (group 2), and Neumifil-treated groups dosed 1 hour before infection (group 3); 3 days, 1 day and 1 hour before infection (group 4); 1 hour before infection, 1 day and 3 days after infection (group 5); 1 day and 3 days post infection (group 6). Each symbol represents total cells for each individual animal per group and each line. Each column represents the group mean and each bar represents standard error of mean (SEM) of n=8 animals. Changes in each treatment group were compared to RSV-infected animals treated with vehicle using Dunnett's one-way analysis of variance.

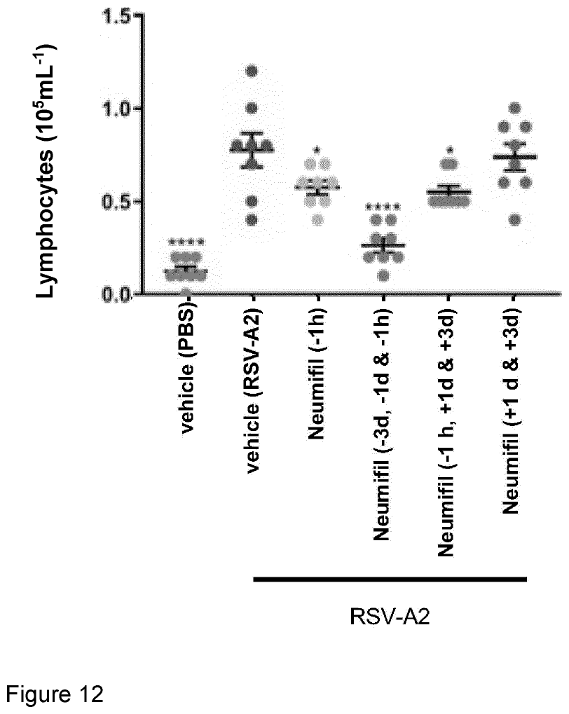

FIG. 12. Scatter plot showing the effect on lymphocyte numbers in bronchoalveolar lavage fluid (BALF) mice infected with RSV-A2 and treated with Neumifil (100 μg, i.n). Groups represent vehicle (PBS) control (group 1), vehicle (RSV-A2) control (group 2), and Neumifil-treated groups dosed 1 hour before infection (group 3); 3 days, 1 day and 1 hour before infection (group 4); 1 hour before infection, 1 day and 3 days after infection (group 5); 1 day and 3 days post infection (group 6). Each symbol represents total cells for each individual animal per group and each line. Each column represents the group mean and each bar represents standard error of mean (SEM) of n=8 animals. Changes in each treatment group were compared to RSV-infected animals treated with vehicle using Dunnett's one-way analysis of variance. * P<0.05, **** P<0.0001.

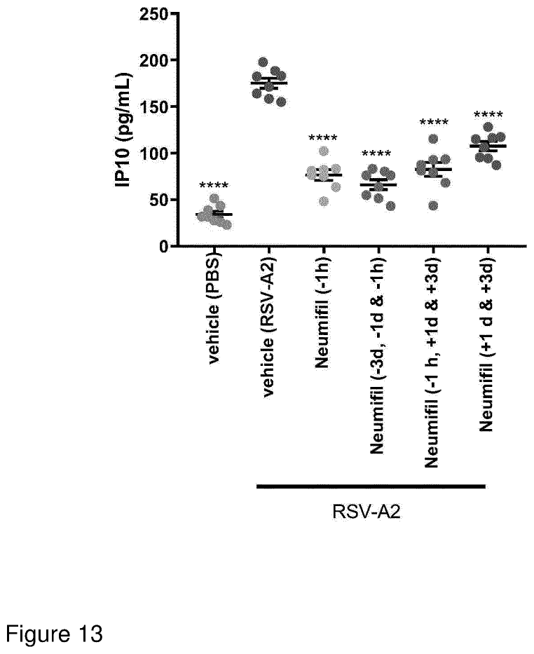

FIG. 13. Scatter plot showing the effect on IP-10 concentrations in bronchoalveolar lavage fluid (BALF) supernatant mice infected with RSV-A2 and treated with Neumifil (100 μg, i.n). Groups represent vehicle (PBS) control (group 1), vehicle (RSV-A2) control (group 2), and Neumifil-treated groups dosed 1 hour before infection (group 3); 3 days, 1 day and 1 hour before infection (group 4); 1 hour before infection, 1 day and 3 days after infection (group 5); 1 day and 3 days post infection (group 6). Each symbol represents the concentration for each individual animal per group and each line. Each column represents the group mean and each bar represents SEM of n=8 animals. Changes in each treatment group were compared to RSV-infected animals treated with vehicle using Dunnett's one-way analysis of variance. **** P<0.0001.

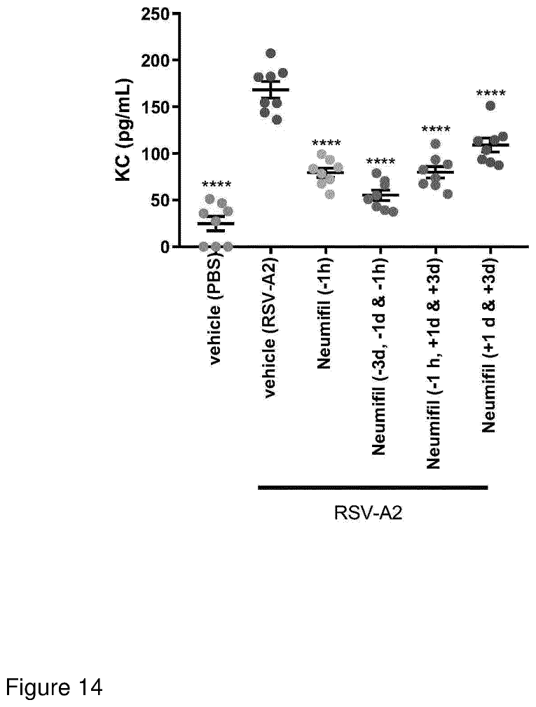

FIG. 14. Scatter plot showing the effect on KC concentrations in bronchoalveolar lavage fluid (BALF) supernatant mice infected with RSV-A2 and treated with Neumifil (100 μg, i.n). Groups represent vehicle (PBS) control (group 1), vehicle (RSV-A2) control (group 2), and Neumifil-treated groups dosed 1 hour before infection (group 3); 3 days, 1 day and 1 hour before infection (group 4); 1 hour before infection, 1 day and 3 days after infection (group 5); 1 day and 3 days post infection (group 6). Each symbol represents the concentration for each individual animal per group and each line. Each column represents the group mean and each bar represents SEM of n=8 animals. Changes in each treatment group were compared to RSV-infected animals treated with vehicle using Dunnett's one-way analysis of variance. **** P<0.0001.

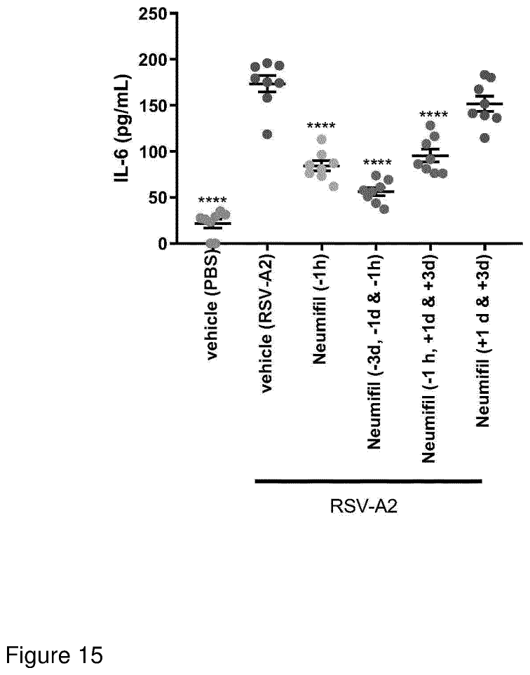

FIG. 15. Scatter plot showing the effect on IL-6 concentrations in bronchoalveolar lavage fluid (BALF) supernatant mice infected with RSV-A2 and treated with Neumifil (100 μg, i.n). Groups represent vehicle (PBS) control (group 1), vehicle (RSV-A2) control (group 2), and Neumifil-treated groups dosed 1 hour before infection (group 3); 3 days, 1 day and 1 hour before infection (group 4); 1 hour before infection, 1 day and 3 days after infection (group 5); 1 day and 3 days post infection (group 6). Each symbol represents the concentration for each individual animal per group and each line. Each column represents the group mean and each bar represents SEM of n=8 animals. Changes in each treatment group were compared to RSV-infected animals treated with vehicle using Dunnett's one-way analysis of variance. **** P<0.0001.

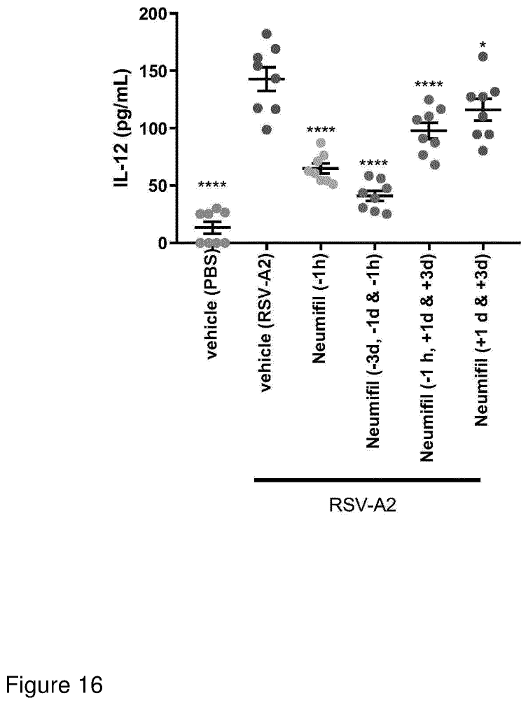

FIG. 16. Scatter plot showing the effect on IL-12 concentrations in bronchoalveolar lavage fluid (BALF) supernatant mice infected with RSV-A2 and treated with Neumifil (100 μg, i.n). Groups represent vehicle (PBS) control (group 1), vehicle (RSV-A2) control (group 2), and Neumifil-treated groups dosed 1 hour before infection (group 3); 3 days, 1 day and 1 hour before infection (group 4); 1 hour before infection, 1 day and 3 days after infection (group 5); 1 day and 3 days post infection (group 6). Each symbol represents the concentration for each individual animal per group and each line. Each column represents the group mean and each bar represents SEM of n=8 animals. Changes in each treatment group were compared to RSV-infected animals treated with vehicle using Dunnett's one-way analysis of variance. * P<0.05, **** P<0.0001.

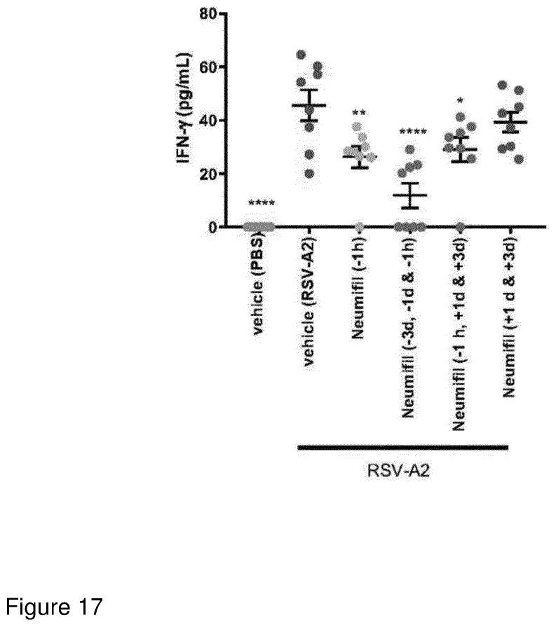

FIG. 17. Scatter plot showing the effect on Interferon-γ concentrations in bronchoalveolar lavage fluid (BALF) supernatant mice infected with RSV-A2 and treated with Neumifil (100 μg, i.n). Groups represent vehicle (PBS) control (group 1), vehicle (RSV-A2) control (group 2), and Neumifil-treated groups dosed 1 hour before infection (group 3); 3 days, 1 day and 1 hour before infection (group 4); 1 hour before infection, 1 day and 3 days after infection (group 5); 1 day and 3 days post infection (group 6). Each symbol represents the concentration for each individual animal per group and each line. Each column represents the group mean and each bar represents SEM of n=8 animals. Changes in each treatment group were compared to RSV-infected animals treated with vehicle using Dunnett's one-way analysis of variance. * P<0.05, ** P<0.01, **** P<0.0001.

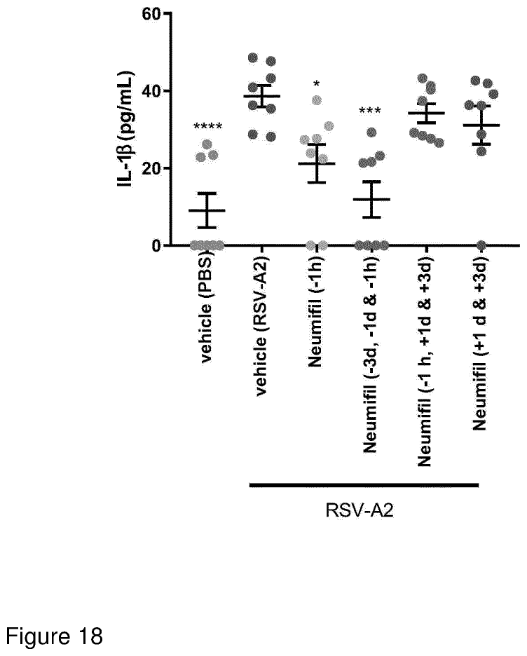

FIG. 18. Scatter plot showing the effect on IL-1β concentrations in bronchoalveolar lavage fluid (BALF) supernatant mice infected with RSV-A2 and treated with Neumifil (100 μg, i.n). Groups represent vehicle (PBS) control (group 1), vehicle (RSV-A2) control (group 2), and Neumifil-treated groups dosed 1 hour before infection (group 3); 3 days, 1 day and 1 hour before infection (group 4); 1 hour before infection, 1 day and 3 days after infection (group 5); 1 day and 3 days post infection (group 6). Each symbol represents the concentration for each individual animal per group and each line. Each column represents the group mean and each bar represents SEM of n=8 animals. Changes in each treatment group were compared to RSV-infected animals treated with vehicle using Dunnett's one-way analysis of variance. * P<0.05, *** P<0.001, **** P<0.0001.

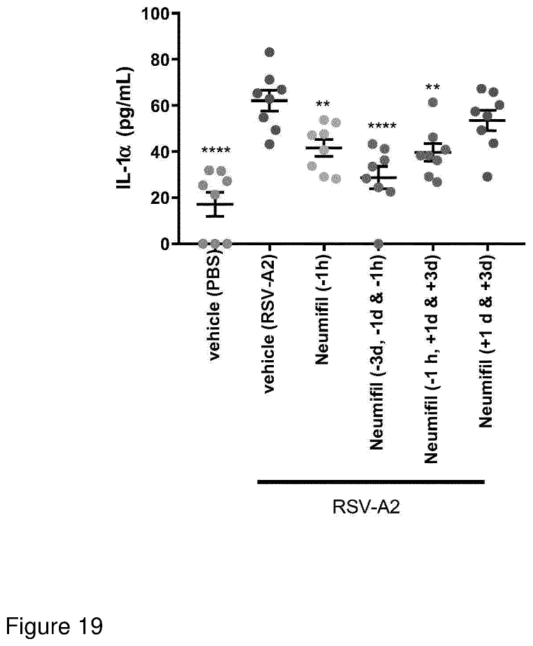

FIG. 19. Scatter plot showing the effect on IL-la concentrations in bronchoalveolar lavage fluid (BALF) supernatant mice infected with RSV-A2 and treated with Neumifil (100 μg, i.n). Groups represent vehicle (PBS) control (group 1), vehicle (RSV-A2) control (group 2), and Neumifil-treated groups dosed 1 hour before infection (group 3); 3 days, 1 day and 1 hour before infection (group 4); 1 hour before infection, 1 day and 3 days after infection (group 5); 1 day and 3 days post infection (group 6). Each symbol represents the concentration for each individual animal per group and each line. Each column represents the group mean and each bar represents SEM of n=8 animals. Changes in each treatment group were compared to RSV-infected animals treated with vehicle using Dunnett's one-way analysis of variance. ** P<0.01, **** P<0.0001.

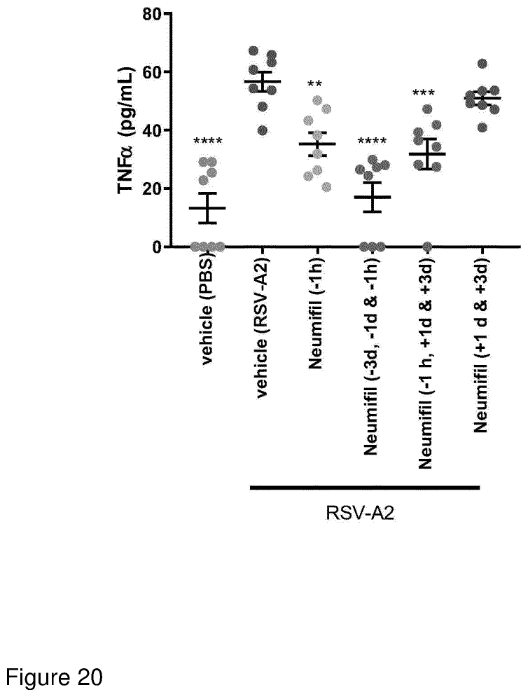

FIG. 20. Scatter plot showing the effect on TNFα concentrations in bronchoalveolar lavage fluid (BALF) supernatant mice infected with RSV-A2 and treated with Neumifil (100 μg, i.n). Groups represent vehicle (PBS) control (group 1), vehicle (RSV-A2) control (group 2), and Neumifil-treated groups dosed 1 hour before infection (group 3); 3 days, 1 day and 1 hour before infection (group 4); 1 hour before infection, 1 day and 3 days after infection (group 5); 1 day and 3 days post infection (group 6). Each symbol represents the concentration for each individual animal per group and each line. Each column represents the group mean and each bar represents SEM of n=8 animals. Changes in each treatment group were compared to RSV-infected animals treated with vehicle using Dunnett's one-way analysis of variance. ** P<0.01, *** P<0.001, **** P<0.0001.

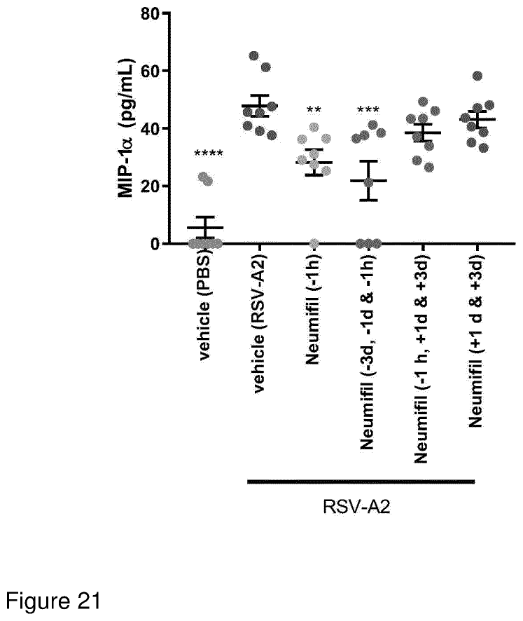

FIG. 21. Scatter plot showing the effect on MIP-la concentrations in bronchoalveolar lavage fluid (BALF) supernatant mice infected with RSV-A2 and treated with Neumifil (100 μg, i.n). Groups represent vehicle (PBS) control (group 1), vehicle (RSV-A2) control (group 2), and Neumifil-treated groups dosed 1 hour before infection (group 3); 3 days, 1 day and 1 hour before infection (group 4); 1 hour before infection, 1 day and 3 days after infection (group 5); 1 day and 3 days post infection (group 6). Each symbol represents the concentration for each individual animal per group and each line. Each column represents the group mean and each bar represents SEM of n=8 animals. Changes in each treatment group were compared to RSV-infected animals treated with vehicle using Dunnett's one-way analysis of variance. ** P<0.01, *** P<0.001, **** P<0.0001.

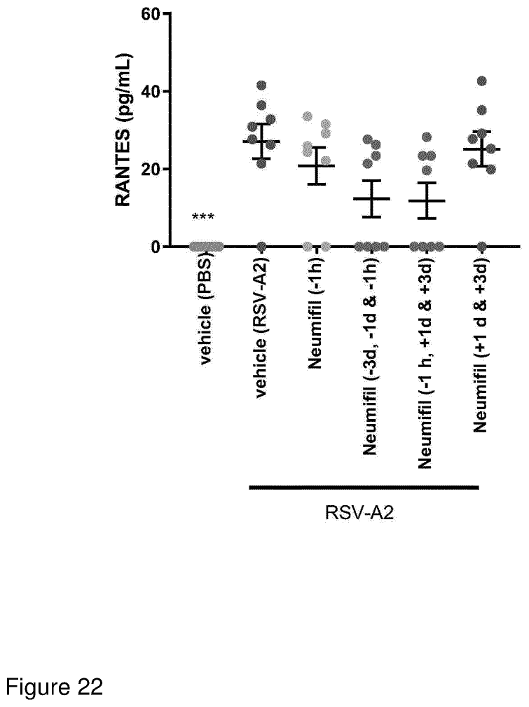

FIG. 22. Scatter plot showing the effect on RANTES concentrations in bronchoalveolar lavage fluid (BALF) supernatant mice infected with RSV-A2 and treated with Neumifil (100 μg, i.n). Groups represent vehicle (PBS) control (group 1), vehicle (RSV-A2) control (group 2), and Neumifil-treated groups dosed 1 hour before infection (group 3); 3 days, 1 day and 1 hour before infection (group 4); 1 hour before infection, 1 day and 3 days after infection (group 5); 1 day and 3 days post infection (group 6). Each symbol represents the concentration for each individual animal per group and each line. Each column represents the group mean and each bar represents SEM of n=8 animals. Changes in each treatment group were compared to RSV-infected animals treated with vehicle using Dunnett's one-way analysis of variance. *** P<0.001.

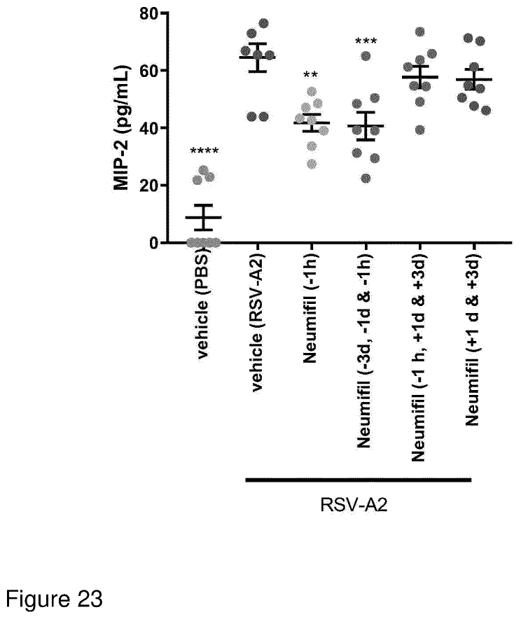

FIG. 23. Scatter plot showing the effect on MIP-2 concentrations in bronchoalveolar lavage fluid (BALF) supernatant mice infected with RSV-A2 and treated with Neumifil (100 μg, i.n). Groups represent vehicle (PBS) control (group 1), vehicle (RSV-A2) control (group 2), and Neumifil-treated groups dosed 1 hour before infection (group 3); 3 days, 1 day and 1 hour before infection (group 4); 1 hour before infection, 1 day and 3 days after infection (group 5); 1 day and 3 days post infection (group 6). Each symbol represents the concentration for each individual animal per group and each line. Each column represents the group mean and each bar represents SEM of n=8 animals. Changes in each treatment group were compared to RSV-infected animals treated with vehicle using Dunnett's one-way analysis of variance. ** P<0.01, *** P<0.001, **** P<0.0001.

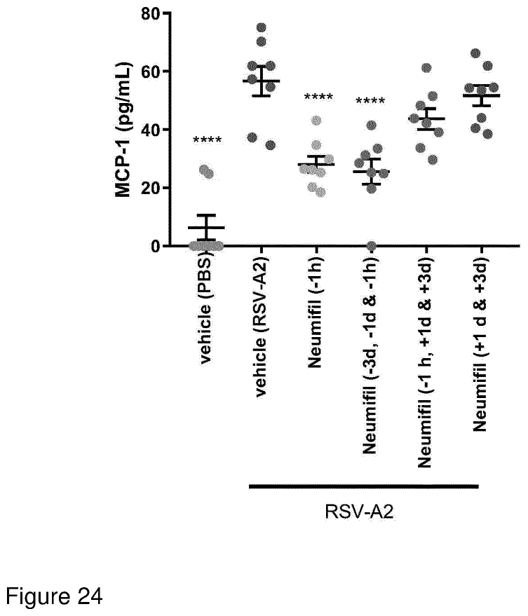

FIG. 24. Scatter plot showing the effect on MCP-1 concentrations in bronchoalveolar lavage fluid (BALF) supernatant mice infected with RSV-A2 and treated with Neumifil (100 μg, i.n). Groups represent vehicle (PBS) control (group 1), vehicle (RSV-A2) control (group 2), and Neumifil-treated groups dosed 1 hour before infection (group 3); 3 days, 1 day and 1 hour before infection (group 4); 1 hour before infection, 1 day and 3 days after infection (group 5); 1 day and 3 days post infection (group 6). Each symbol represents the concentration for each individual animal per group and each line. Each column represents the group mean and each bar represents SEM of n=8 animals. Changes in each treatment group were compared to RSV-infected animals treated with vehicle using Dunnett's one-way analysis of variance. **** P<0.0001.

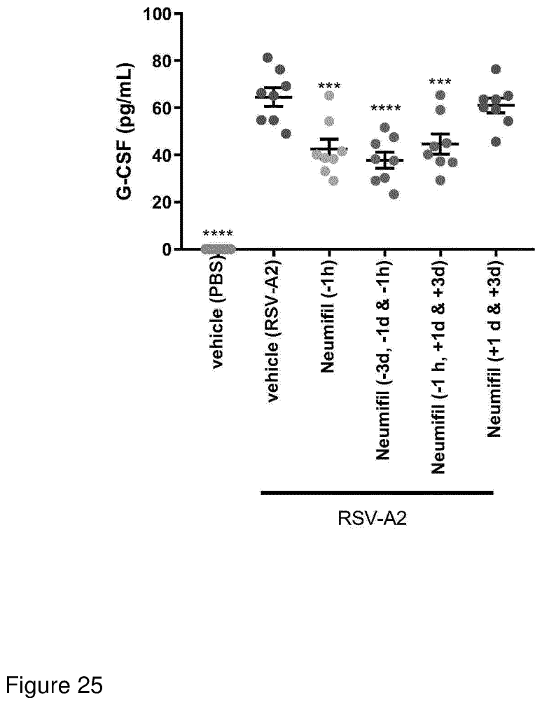

FIG. 25. Scatter plot showing the effect on G-CSF concentrations in bronchoalveolar lavage fluid (BALF) supernatant mice infected with RSV-A2 and treated with Neumifil (100 μg, i.n). Groups represent vehicle (PBS) control (group 1), vehicle (RSV-A2) control (group 2), and Neumifil-treated groups dosed 1 hour before infection (group 3); 3 days, 1 day and 1 hour before infection (group 4); 1 hour before infection, 1 day and 3 days after infection (group 5); 1 day and 3 days post infection (group 6). Each symbol represents the concentration for each individual animal per group and each line. Each column represents the group mean and each bar represents SEM of n=8 animals. Changes in each treatment group were compared to RSV-infected animals treated with vehicle using Dunnett's one-way analysis of variance. *** P<0.001, **** P<0.0001.

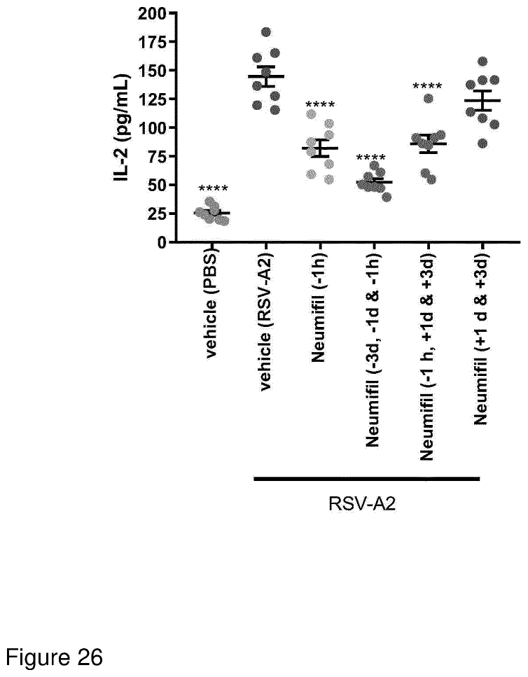

FIG. 26. Scatter plot showing the effect on IL-2 concentrations in bronchoalveolar lavage fluid (BALF) supernatant mice infected with RSV-A2 and treated with Neumifil (100 μg, i.n). Groups represent vehicle (PBS) control (group 1), vehicle (RSV-A2) control (group 2), and Neumifil-treated groups dosed 1 hour before infection (group 3); 3 days, 1 day and 1 hour before infection (group 4); 1 hour before infection, 1 day and 3 days after infection (group 5); 1 day and 3 days post infection (group 6). Each symbol represents the concentration for each individual animal per group and each line. Each column represents the group mean and each bar represents SEM of n=8 animals. Changes in each treatment group were compared to RSV-infected animals treated with vehicle using Dunnett's one-way analysis of variance. **** P<0.0001.

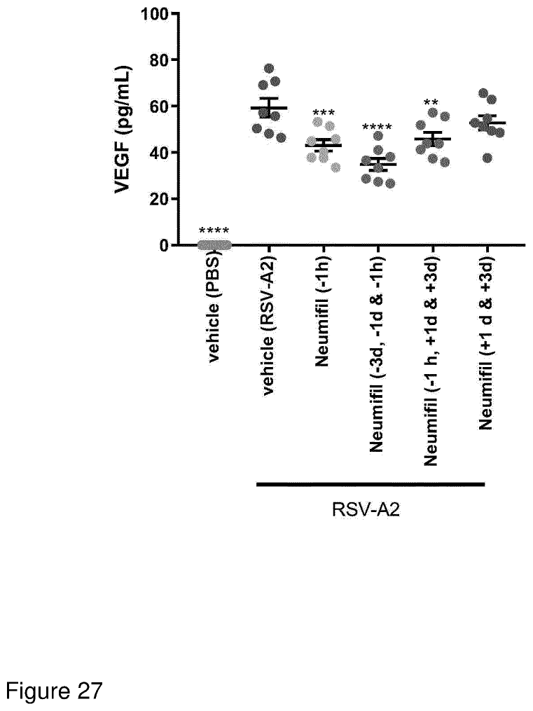

FIG. 27. Scatter plot showing the effect on VEGF concentrations in bronchoalveolar lavage fluid (BALF) supernatant mice infected with RSV-A2 and treated with Neumifil (100 μg, i.n). Groups represent vehicle (PBS) control (group 1), vehicle (RSV-A2) control (group 2), and Neumifil-treated groups dosed 1 hour before infection (group 3); 3 days, 1 day and 1 hour before infection (group 4); 1 hour before infection, 1 day and 3 days after infection (group 5); 1 day and 3 days post infection (group 6). Each symbol represents the concentration for each individual animal per group and each line. Each column represents the group mean and each bar represents SEM of n=8 animals. Changes in each treatment group were compared to RSV-infected animals treated with vehicle using Dunnett's one-way analysis of variance. **P<0.01, *** P<0.001, ****P<0.0001.

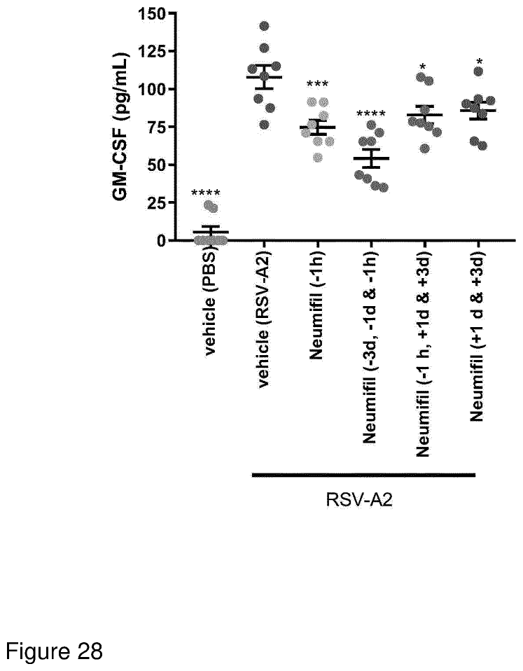

FIG. 28. Scatter plot showing the effect on GM-CSF concentrations in bronchoalveolar lavage fluid (BALF) supernatant mice infected with RSV-A2 and treated with Neumifil (100 μg, i.n). Groups represent vehicle (PBS) control (group 1), vehicle (RSV-A2) control (group 2), and Neumifil-treated groups dosed 1 hour before infection (group 3); 3 days, 1 day and 1 hour before infection (group 4); 1 hour before infection, 1 day and 3 days after infection (group 5); 1 day and 3 days post infection (group 6). Each symbol represents the concentration for each individual animal per group and each line. Each column represents the group mean and each bar represents SEM of n=8 animals. Changes in each treatment group were compared to RSV-infected animals treated with vehicle using Dunnett's one-way analysis of variance. * P<0.05, *** P<0.001, ****P<0.0001.

### METHODS AND RESULTS

### Example 1

### Sp2CBMTD: Prediction of Immunogenic Regions

### Nordic Biopharma in Silico Screen

The in silico T-cell epitope screening identified four significant and two borderline immunogenic clusters:

### Significant:

| Domain | Residue range | Sequence |
|---|---|---|
| SpCBM | 245 to 254 | GVLSRTSLRS |
| PaTD | 340 to 349 | WFSVSSNSLY |
| PaTD | 351 to 359 | LSHGLQRSP |
| PaTD | 398 to 406 | GSLNIRLGT |

### Borderline:

| Domain | Residue range | Sequence |
|---|---|---|
| SpCBM | 167 to 178 | FYNLFSVSSATK |
| SpCBM | 239 to 251 | VRLYVNGVLSRTS |

### ProImmune Human Donor T-Cell Proliferation Assay

The ProImmune study highlighted two regions of high antigenicity and two regions of moderate antigenicity:

### High Antigenicity:

| Domain | Residue range | Sequence |
|---|---|---|
| SpCBM | 236 to 250 | KGRVRLYVNGVLSRT |
| PaTD | 392 to 406 | GAQVEVGSLNIRLGT |

### Moderate Antigenicity:

| Domain | Residue range | Sequence |
|---|---|---|
| SpCBM | 167 to 181 | FYNLFSVSSATKKDE |
| PaTD | 338 to 352 | SDWFSVSSNSLYTLS |

### ProPred in Silico Analysis

A further in silico tool, the online ProPred server⁴, was also used. The output of the ProPred server is shown in FIG. 2. The relative positions of the Nordic Biopharma/ProImmune epitopes are also highlighted and indicate reasonable agreement between the three methods. In addition to the epitopes listed above, ProPred strongly predicted another immunogenic epitope in the SpCBM domain:

| Domain | Residue range | Sequence |
|---|---|---|
| SpCBM | 286 to 294 | IRNLTVYNR |

### Mutations in the Individual CBM and TD Domains

To guide the design of mutations that might reduce immunogenicity, ProPred was used to test the effect of changing each residue in these peptides to every alternative residue. Those that gave the greatest reduction in predicted number of allele binders were noted. As the crystal structure of both the SpCBM and TD domains are known, these mutations were also modelled to reduce the likelihood of introducing mutations that would obviously disrupt the protein structure.

Initially, nine single mutations in SpCBM and four single mutations in PaTD were introduced and are listed below (‘Im’ is short for immunogenicity mutant):

| (SpCBM) variants | Mutation | (PaTD) variants | Mutation |
|---|---|---|---|
| WTSp | — | WTTD | — |
| Im15 | Y168W | Im24 | S342D |
| Im16 | L170A | Im25 | S345D |
| Im17 | L170T | Im26 | L348D |
| Im18 | V173G | Im27 | R403K |
| Im19 | V239A | | |
| lm20 | V239T | | |
| Im21 | V246G | | |
| Im22 | I286A | | |
| Im23 | Y292E | | |

Note:
Im1 to Im14 (not shown) were introduced by mutagenesis into a non-codon optimized background, before the Prolmmune data were available.

### Synthesis of WT and Mutated Constructs

The genes encoding WT SpCBM, WT PaTD and the variants Im15 to Im27 were codon optimized for E. coli expression and synthesized by GeneArt. The genes were then cloned in-house into the pHISTEV vector for expression as 6His-tagged proteins.

### Expression and Biophysical Characterization

An initial expression test was performed to assess solubility. The results show that all were expressed, but not all were soluble (FIG. 3). Note: solubility (or a lack thereof) is not necessarily a predictor of utility. One of skill will appreciate that when manufacturing or producing proteins, certain processes require the use of insoluble material as this is readily purified (from inclusion bodies and the like). Downstream protocols may then re-engineer proteins to modulate features such as solubility.

Results of the expression test show that:
Im16 (L170A) is insoluble or very poorly soluble
Im25 (TD, S345D) is insoluble
Im15 (Y168W) and Im17 (L170T) have reduced solubility
Im18 (V173G) and Im22 (I286A) are slightly reduced.
The remainder show soluble expression.

The 13 soluble proteins were expressed in E. coli and purified by immobilized metal affinity chromatography (IMAC), followed by TEV digestion to remove the 6His-tag, then reverse IMAC and size exclusion chromatography (SEC).

Ten purified domains (WTSp, Im19, Im20, Im21, Im22, Im23, WTTD, Im24, Im26 and Im27) were further characterized by:

(i) Thermofluor to measure melting temperature (Tm)

(ii) Near UV circular dichroism (CD) to compare tertiary structures to WT

(iii) Dynamic light scattering (DLS) to check oligomeric state in solution

(iv) Surface plasmon resonance (SPR) to measure binding affinity to sialyllactose

(v) Measurement of IL-8 cytokine stimulation

The results are summarized in Table 1.

TABLE 1
Qualitative summary of the biophysical characterizations of the WT domains and their
variants. Colour coding is from green to red (including green’, orange and yellow), where green
indicates that the variant closely resembles its WT counterpart for that particular characteristic
and pale green (green’) or yellow indicate increasing degrees of differences. Red or orange
indicate significant differences.

| | | | | Tm +/− | NearUV | | | Cytokine |
|---|---|---|---|---|---|---|---|---|
| Name | Mutation | Solubility | Purification | 6SL | CD | DLS | Biacore | stimulation |
| WTSp | — | Green | Green | Green | Green | Green | Green | Green |
| Im15 | Y168W | Yellow | Orange | Red | N/A | N/A | N/A | N/A |
| Im16 | L170A | Red | N/A | N/A | N/A | N/A | N/A | N/A |
| Im17 | L170T | Yellow | Orange | N/A | N/A | N/A | N/A | N/A |
| Im18 | V173G | Green’ | Orange | N/A | N/A | N/A | N/A | N/A |
| Im19 | V239A | Green | Green | Green | Green | Green | Green | N/D |
| Im20 | V239T | Green | Green | Green’ | Green’ | Green | Green | N/D |
| Im21 | V246G | Green | Green | Green’ | Green | Green | Green | Green |
| Im22 | I286A | Green’ | Green | Green’ | Green | Green | Green | Green |
| Im23 | Y292E | Green | Green | Green’ | Green’ | Green | Yellow | Yellow |
| Im24 | S342D | Green | Green | Green | Green | Green | | |
| Im25 | S345D | Red | N/A | N/A | N/A | N/A | | |
| Im26 | L348D | Green | Green | Green’ | Green | Green | | |
| Im27 | R403K | Green | Green | Green | Green | Green | | |
| WTTD | — | Green | Green | Green | Green | Green | | |

N/A: these characterizations were not performed due to poor solubility/purity of the protein.
N/D: not determined.

### Sp Peptide 167-181:

Im15, Im16, Im17, Im18 are all insoluble or poorly soluble (as stated, this does not necessarily impact on protein utility). These are in the ‘moderately’ antigenic region 167-181 (FYNLFSVSSATKKDE). This region is clearly very sensitive to change.

Earlier results show that M156F, which sits adjacent to L170 (and I286), increases Tm by ˜4° C.

This could therefore be combined with L170T. M156F does not increase predicted immunogenicity.

M185I increases Tm by 5° C., and lies parallel to L170 (FIG. 4). This mutation could also be included. Note that, like M156F, M185I does not increase predicted immunogenicity but slightly reduces the number of predicted allele binders.

### Sp Peptide 236-250:

Im19, Im20, Im21 all behave similarly to WT. These are in the ‘highly’ antigenic region 236-250 (KGRVRLYVNGVLSRT).

Im19 (V239A) was chosen over the threonine mutation (Im20, V239T). There is no difference in predicted immunogenicity but Im19 is a closer match to WT Thermofluor Tm and Near UV spectrum. This would be combined with Im21 (V246G).

### Sp Peptide 286-294:

Im22 (I286A) is broadly similar to WT while Im23 (Y292E) appears to exhibit reduced ligand affinity. This region, 286-294 IRNLTVYNR, was not flagged up by ProImmune but is strongly predicted by ProPred to be immunogenic.

There is some indication that Im22 has lower Tm than WT. This residue is adjacent to M156 so may behave differently if M156F was included.

### TD Peptide 338-352:

Im24 (S342D) and Im26 (L348D) show similar characteristics to the WT trimerization domain, but with some suggestion of reduced Tm in Im26. These are in the ‘moderately’ antigenic region 338-352 SDWFSVSSNSLYTLS. The WT sequence was predicted to bind 9 alleles, while Im24 predicts 2 alleles and a Im24/Im26 double mutant predicts 1 allele.

### TD Peptide 392-406:

Im27 (R403K) is similar to WT. It is part of the ‘highly’ antigenic region 392-406 GAQVEVGSLNIRLGT. Predicted alleles are reduced from 21 to 3 when this mutation is introduced.

### Synthesis of Multiple Mutation Combinations Im28-34

The following mutations were introduced:

i) M156F/L170T

ii) M156F/L170T/M185I: In ProPred, alleles predicted for this region are reduced from 31 in the WT to 19 for this combination.

iii) V239A/V246G: In ProPred, alleles for this region are reduced from 44 to 3.

iv) I286A/Y292E: In ProPred, alleles are reduced from 41 to 1.

v) V239A/V246G/I286A/Y292E combines the previous two doubles.

vi) M156F/L170T/M185I/V239A/V246G/I286A/Y292E combines all the Sp mutations

vii) TD: S342D/L348D/R403K: Predicted alleles are reduced from 9 to 1 for TD peptide 338-352 and alleles for peptide TD peptide 392-406 are reduced from 21 to 3. This triple mutant combines all the TD mutants. They are all surface exposed and distal to the N-terminal end of TD, so would not be expected to interfere with SpCBM in the hexamer form.

The constructs are named Im28 to Im34:

| (SpCBM) variant | Mutations |
|---|---|
| Im28 | M156F/L170T |
| Im29 | M156F/L170T/M185I |
| lm30 | V239A/V246G |
| Im31 | I286A/Y292E |
| Im32 | V239A/V246G/I286A/Y292E |
| Im33 | M156F/L170T/M185I/V239A/V246G/I286A/Y292E |
| (PaTD) variant | Mutation |
| Im34 | S342D/L348D/R403K |

### 2.5 Expression and Biophysical Characterization of Im28-Im34

As with the single mutations, the combinations Im28 to Im34 were synthesized by GeneArt and subcloned into pHISTEV for expression analysis. A nickel bead pull-down on the His-tagged soluble extract was also performed (FIG. 5).

### Hexameric Forms

### Design of Hexameric Constructs HEX1 to HEX17

Genes encoding the hexameric forms (called HEX1 to HEX17) were synthesized by GeneArt:

### Sp2CBMTD

| variant | Mutations |
|---|---|
| HEX1 | CBM1(L170T V239A V246G I286A Y292E)-CBM2(L170T |
| | V239A V246G I286A Y292E)-TD (S342D L348D R403K) |
| HEX2 | CBM1(V239A V246G I286A Y292E)-CBM2(V239A V246G |
| | I286A Y292E)- TD (S342D R403K) |
| HEX3 | CBM1(V239A V246G I286A)-CBM2(V239A V246G I286A)- |
| | TD (S342D R403K) |
| HEX4 | CBM1(V239A V246G)-CBM2(V239A V246G)-TD (S342D) |
| HEX5 | CBM1(V239A V246G)-CBM2(V239A V246G)-TD(R403K) |
| HEX6 | CBM1(V239A V246G)- CBM2(V239A V246G)-TD (S342D |
| | R403K) |
| HEX17 | CBM1(V239A V246G A162P)- CBM2(V239A V246G A162P)- |
| | TD (S342D R403K) |

The hexameric forms were synthesized in two parts to avoid problems associated with synthesising repeat sequences in the tandem CBM copies. The first gene covered the first CBM and the second part encompassed the second CBM plus the TD. These could then be simultaneously cloned into pHISTEV to create the Sp2CBMTD construct that trimerizes upon expression.

The first hexamer, HEX1, contained the mutations L170T/V239A/V246G/I286A/Y292E in the CBMs and S342D/L348D/R403K in the TD.

The solubility data of the individual domains indicated that HEX1 was unlikely to be soluble (again, not necessarily a reflection on the utility of the molecule); a further construct, HEX3, was synthesized. Note that HEX2 contained the same mutations as HEX3, but with the addition of Y292E.

HEX3 was synthesized and subcloned into the pHISTEV vector. Expression was insoluble under all conditions tested (varying temperature, IPTG concentration, cell density at induction, with or without heat shock). The CBM-only domain containing the same three mutations (V239A V246G I286A) is soluble. A double mutant (V239A V246G) behaves very similarly to WT. Therefore, further variants (HEX4, HEX5 and HEX6) were designed and constructed by PCR/ligations, which exclude I286A and contain either one or both of the TD mutations.

During the work on HEX6 a number of other versions were designed containing different combinations of the HEX6 mutations (numbered HEX7 to HEX16; not characterised).

HEX17 contains the HEX6 mutations with an additional A162P mutation. This proline mutation has been shown to increase the single CBM Tm by 3-4° C. The proline mutation is not near the other mutations, the N- or C-termini or the ligand binding site.

### Characterization of the Hexameric Variants

The expression, purification and characterization results are shown in Table 2. Based on these results, HEX6 and HEX17 were taken forward. The positions of the HEX17 mutations on the hexamer are shown in FIG. 6.

TABLE 2
Qualitative summary of the biophysical characterizations of the hexameric
Sp2CBMTD variants. Colour coding is from green to red, where green indicates that the
variant closely resembles its WT counterpart for that particular characteristic and pale green
(green’) or yellow indicate increasing degrees of differences. Red or orange indicate significant
differences.

| | | | | | NearUV | | IL-8 |
|---|---|---|---|---|---|---|---|
| Name | Mutations | Solubility | Purification | Thermostability | CD | Biacore | assay |
| Hex1 | L170T/V239A/V246G/I286A/Y292E/ S342D/L348D/R403K | Red | N/A | N/A | N/A | N/A | N/A |
| Hex2 | V239A/V246G/I286A/Y292E/S342D/R403K (designed but not made) | | | | | | |
| Hex3 | V239A/V246G/I286A/S342D/R403K | Red | N/A | N/A | N/A | N/A | N/A |
| Hex4 | V239A/V246G/S342D | Yellow | Red | N/A | N/A | N/A | N/A |
| Hex5 | V239A/V246G/R403K | Green | Yellow | Yellow | N/A | N/A | N/A |
| Hex6 | V239A/V246G/S342D/R403K | Green | Green | Yellow | Green | Green | Yellow |
| Hex7 | Note: These constructs are different combinations of the Hex6 | | | | | | |
| to 16 | mutations and were designed as a back-up in case Hex6 failed | | | | | | |
| Hex17 | A162P/V239A/V246G/S342D/R403K | Green | Green | Green’ | Green | Green | reduced |
| | | | | | | | IL-8 |

N/A: these characterizations were not performed due to poor solubility/purity of the protein.
N/D: not determined.

### Example 2: Inflammatory Mediators

Aim: To Measure the Innate Immune Response of mCBM-Treated Human Lung Epithelial Cells (A549) by Analysing Levels of Inflammatory Mediators Over Time.

Administration of Sp2CBMTD to mammalian cells stimulated a pro-inflammatory response both in vitro and in vivo^{1,2}. To determine whether this was still observed with modified hexameric sialic acid binding molecules, mammalian A549 cells were stimulated by the addition of 10 μg of biologic (Sp2CBMTD (aka SpOrig), HEX6 (i.e. a sialic acid binding molecule comprising 3×HEX6 units) or HEX17 (i.e. a sialic acid binding molecule comprising 3×HEX17 units) and cell culture medium was harvested at specific time-points post administration. The concentrations of inflammatory mediators were measured both by ELISA and a multiplex assay.

Human IL-8 (benchmark cytokine for the study) response using a human 1× Mouse CXCL1/KC Quantikine ELISA Kit (R&D BioSystems). The concentration levels of IL-8 from stimulated A549 cells are shown in FIG. 7. It is evident that when A549 cells are stimulated with the modified hexamer HEX17, IL-8 levels are significantly lower than when compared to Sp2CBMTD (aka SpOrig) stimulated cells.

Inflammatory mediator response using a Human Cytokine 12-plex Assay (Bio-Plex Pro™, Bio-Rad). FIG. 8 demonstrates the analysis of 12 inflammatory mediators from culture medium after A549 cell stimulation by Sp2CBMTD (WT, aka SpOrig), HEX6 and HEX17 (variants) at specific time points (6 h, 24 h, 48 h). Prior to analysis, samples were thawed and diluted 1:4 in PBS before using a human HS Cytokine-12 plex assay (R&D Systems). The data indicates that:
HEX17 affects the levels of almost all the cytokines tested compared to SpOrig and HEX6. There is a significant reduction in observed concentration (pg/ml) with analytes IL-6, IL-8, GM-CSF and IFN-gamma at 48 h when compared to SpOrig and HEX6.
When compared to control at 48 h, HEX17 appears to cause an increase in the level of all cytokines tested with the exception of IL-5, and VEGF (yet to be confirmed).
HEX6 only showed reduced IL-6 stimulation compared to SpOrig at 48 h.

### Example 3

The objective of this study was to evaluate the effect of HEX17(aka “Neumifil”) on RSV virus replication, cell accumulation and biomarkers on day 4 post infection in mice.
Mice were treated with either vehicle or Neumifil (100 μg, i.n.) prophylactically on day 3, day 1 and 1 hour pre RSV-A2 infection or 1 hour pre RSV-A2 infection, therapeutically on day 1 and day 3 post RSV-A2 infection or with a combined prophylactic and therapeutic regimen 1 hour pre RSV-A2 infection and day 1 and day 3 post infection.
RSV-A2 infected mice that were vehicle treated showed a significant increase in lung viral load at 4 days post infection when compared to PBS-challenged animals. This was accompanied by a significant pulmonary inflammation as demonstrated by increased cell numbers in bronchoalveolar lavage fluid (BALF), especially neutrophils and lympthocytes. Pro-inflammatory cytokines were also significantly elevated in the BALF fluid.
Treatment with Neumifil in RSV-A2 infected mice resulted in a significant protective effect as evidenced by a reduction in lung tissue viral load and reduced inflammatory cell influx and cytokine concentrations in BALF. This was particularly evident in animals receiving a prophylactic dosing regimen in which animals receiving doses day 3, day 1 and 1 hour pre-infection were offered the greatest protection.
Data from this study demonstrates that Neumifil has a possible protective and therapeutic effect in treating RSV-A2 infection.

### Test Vehicle

Supplied by Pneumagen. Quantity: 10×1 mL aliquots of phosphate buffered saline (10×) diluted to 1× using MilliQ water pH 7.2. Manufacturer: Life Technologies. Product Number: 70013-016. Storage: Room temperature or 20° C.

### Test Agent (HEX17: Neumifil)

Supplied by Pneumagen (Batch Number: 20190410A; Certificate Number: PGN0030/290419). Quantity: 11×100 μL aliquots of protein at a concentration of 10 mg/mL storage: −20° C.

### RSV Infection

Non-fasted mice (female BALB/c, 17-20 g) were weighed, individually identified on the tail with a permanent marker and then infected intranasally with RSV or virus diluent (DMEM, 2% v/v FCS, 12.5% w/v Sucrose) under isoflurane (5% in O₂) anesthesia. The A2 strain of RSV (50 μL of 5×10⁶ PFU) was instilled into each nostril in a drop-wise fashion, alternating between the two until a volume of 50 μL has been delivered. At the outset of the study, the A2 strain of RSV was back-titrated to determine viability of the virus.

### Test Agent Formulation

Throughout the study the test vehicle and test agent were formulated as detailed below.

### For Test Vehicle (PBS) Preparation:

On each day of dosing, an aliquot (1 mL vial) of the test vehicle was diluted from 10× to 1× to a volume of 10 mL using endotoxin-free water. 1×PBS was then used for Neumifil dilution and also for dosing the vehicle control group.

### For Test Agent (Neumifil) Preparation:

On each day of dosing, an aliquot of Neumifil (100 μL per vial at 10 mg/mL) was thawed and transferred into a new sterile 0.5 mL Eppendorf tube and centrifuge at 13,000 rpm for 5 min. The supernatant was transferred into new sterile 1.5 mL Eppendorf tubes and diluted to a volume of 400 μL using 1×PBS to formulate a concentration of 2.5 mg/mL (equivalent to 100 μg/40 μL). Neumifil tubes were clearly labelled and kept at RT. Prior to dosing, the formulation contents were gently mixed using a pipette, avoiding bubbles.

A new vial of Neumifil or vehicle was used for each group and day of dosing. Remaining formulations (both diluted and non-diluted Neumifil) were stored at −20° C. and shipped back to Pneumagen at the end of the study.

### Dosing

Test agent (Neumifil) or vehicle (PBS) were administered intranasally (40 μL) as the first dose either 3 days before infection or 1 hour before infection or 1 day after infection (see dosing table below for more detail). First dose of 40 μL PBS (vehicle) will be given 1 hour before infection for both vehicle control (group 1) and virus only control (group 2).

### Treatment Group Dosing

| Grp | Day −3 | Day −1 | Hour −1 | Hour 0 | Day 1 | Day 2 | Day 3 |
|---|---|---|---|---|---|---|---|
| 1 | | | PBS | PBS | PBS | | PBS |
| | | | (Vehicle) (i.n. | | | | |
| | | | dosing.) | | | | |
| 2 | | | PBS | RSV-A2 | PBS | | PBS |
| | | | (Vehicle) (i.n. | 5 X 10⁶ PFU | | | |
| | | | dosing.) | | | | |
| 3 | | | Neumifil | RSV-A2 | | | |
| | | | (100 μg, | 5 X 10⁶ PFU | | | |
| | | | i.n.dosing.) | | | | |
| 4 | Neumifil | Neumifil | Neumifil | RSV-A2 | | | |
| | (100 μg, | (100 μg, | (100 μg, | 5 X 10⁶ PFU | | | |
| | i.n.dosing.) | i.n.dosing.) | i.n.dosing.) | | | | |
| 5 | | | Neumifil | RSV-A2 | Neumifil | | Neumifil |
| | | | (100 μg, | 5 X 10⁶ PFU | (100 μg, | | (100 μg, |
| | | | i.n.dosing.) | | i.n.dosing.) | | i.n.dosing.) |
| 6 | | | | RSV-A2 | Neumifil | | Neumifil |
| | | | | 5 X 10⁶ PFU | (100 μg, | | (100 μg, |
| | | | | | i.n.dosing.) | | i.n.dosing.) |

### Clinical Evaluation

Clinical signs for each animal were recorded once a day from Day −3. These included details on piloerection, respiration, activity, body posture, ocular/nasal discharge, body condition and ataxia. These variables were scored with the following severity bands:
0.—Normal
1.—Mild
2.—Laboured
3.—Severe (Cull point)

Daily measurement of each animal's body weight was also recorded from Day −3.

Animals showing two or more of any of the limiting clinical signs (based on Home Office guidelines) equivalent to the protocol severity limit, were removed from the study and were culled by a schedule 1 method (cervical dislocation) at the establishment. Where an animal reached the limit of either or both of the first two signs with or without any other signs, it was removed from the study and culled by a schedule 1 method at the establishment.

Body weight loss greater than 20% of the highest measured individual body weight.
Food and water consumption less than 40% of normal for 3 days or anorexia (total inappetence for 72 hrs).
Marked piloerection with other signs of dehydration such as skin tenting.
Unresponsive to activity and provocation.
Hunched persistently (frozen).
Distressed—persistent vocalization.
Oculo-nasal discharge persistent and copious.
Laboured respiration.
Persistent tremors.
Persistent convulsions.

In this study, no animals were removed from the study on grounds of welfare issues.

### Sample Collection

4 days after infection, all animals were overdosed intraperitoneally with pentobarbitone, and blood samples were collected by venepuncture into Eppendorf-tubes (0.5 mL). Each sample was mixed gently and kept at room temperature for 30 min to allow clotting, before centrifugation (1500 rpm, 10 min at 4° C.) to prepare the serum. 2 aliquots (50 μL) of each sample were stored at −80° C. Immediately after collecting the blood, the trachea was isolated by a midline incision in the neck and separation of the muscle layers. A small incision was made into the trachea and a plastic cannula was inserted and secured in place with a suture. The airway was then lavaged by flushing out the lungs using 1 mL of phosphate buffered saline. This procedure was then repeated until the recovered volume was 1.6 mL. The isolated BALF was then be centrifuged at 1500 rpm for 10 mins at 4° C. and the supernatant was aliquoted (400 μL) at −80° C. for future cytokine analysis. The cell pellets were then re-suspended in 0.8 mL of 0.2% w/v NaCl to induce haemolysis of any erythrocytes. After isotonization with the same volume of 1.6% w/v NaCl, the BAL cells were analysed for total and differential numbers.

Total and differential cell counts of the BAL fluid samples were measured using a XT-2000iV analyser (Sysmex). Results are expressed as cells/mL (total and differential). Cell types differentially classified will be neutrophils, eosinophils or mononuclear cells (macrophages and lymphocytes).

### Lung Tissue Removal

Following BALF collection, the left and right lung lobes were removed and separated from each animal and homogenised in the volume of ×10 gram-lung weight (if 0.11 g, use 1.1 mL) of ice-cold Dulbecco's modified Eagles medium (DMEM containing 1% w/v BSA and 25% w/v sucrose) for 2×20 second bursts. The homogenate was transferred into a sterile tube and spun at 4° C. at 2000 rpm for 5 minutes. The clarified homogenate was then transferred to a chilled cryovial and snap frozen in liquid nitrogen and stored at −80° C.

### Plaque Assay

HEp2 cells were grown in 24-well plates prior to infection in DMEM containing 10% v/v FBS until they attain 100% confluency. Lung homogenates were thawed out at room temperature and ten-fold serial dilutions were prepared in serum-free DMEM. The growth medium from HEp2 cells was aspirated and replaced with 300 μL of serially diluted lung homogenate (along with stock RSV only positive control) and left to infect at 37° C./5% CO₂ for four hours. The infectious media was then aspirated and replaced with 500 μL Plaque Assay Overlay (1% w/v methylcellulose in MEM, 2% v/v FBS, 1% w/v pen/strep, 0.5 μg/ml amphotericin B), and left for 7 days at 37° C./5% CO₂. Cells were fixed with ice-cold methanol for 10 minutes after which they were washed twice with sterile PBS. Anti-RSV F-protein antibody [2F7] was diluted to a 1:150 concentration in blocking buffer (5% w/v powdered milk (Marvel) in 0.05% v/v PBS-Tween 20) and 150 μL were added to cells for 2 hours at room temperature with shaking. Cells were washed 2× using PBS before 150 μl of secondary antibody (goat anti-mouse/HRP conjugate) diluted 1:400 in blocking buffer were added to cells for 1 hour at room temperature, with shaking. The secondary antibody solution was removed and cells were washed twice with PBS before the metal-enhanced development substrate DAB will be prepared in ultra-pure water (according to manufacturer's instructions). Each well received 150 μL of development substrate until plaques are visible. Plaques were counted by eye and confirmed using light microscopy, allowing the calculation of plaque-forming units per mL.

### Biomarker Analysis

Cytokine levels (see below for details of cytokines evaluated) of BALF supernatant were measured in duplicate using magnetic multiplex assays as per the manufacturers' instructions. Levels were measured using a Magpix system (Luminex Corp.)

Data are reported as cytokine (pg/mL), mean±S.E.M. (standard error of the mean).

Mouse cytokine magnetic 23 plex
panel (Biotechne)
GM-CSF
IFN-γ
IL-1β
IL-2
IL-4
IL-5
IL-6
IL-10
IL-12p40/p70
TNFα
IL-1α
IL-13
IL-17
IP-10
KC
MCP-1
G-CSF
MIP-1α
VEGF
RANTES
MIP-2
MIP-1β
IL-33

### Data Analysis

Data are reported as total and differential number of cells per mL of BALF, cytokine concentration (pg/mL) or plaque forming units (pfu) per treatment group, ±(standard error of the mean).

Inter-group deviations were statistically analyzed by a one-way analysis of variance (ANOVA). In the case of significant difference in the mean values among the different levels of treatment, comparisons versus the vehicle group will be carried out using the Dunnett's test. In case the equal variance test fails, a Kruskal-Wallis one-way analysis of variance on ranks followed by a Dunn's test will be proposed. p<0.05 were considered statistically significant.

### CONCLUSIONS

Prophylactic treatment with Neumifil (HEX17) in RSV-A2 challenged mice resulted in a significant protective effect as evidenced by a reduction in lung tissue viral load. A strong effect was seen in mice which received a regimen of three doses of Neumifil on day 3, day 1 and 1 hour pre-challenge. Also, mice that received a single prophylactic dose 1 hour pre-challenge showed a statistically significant reduction in viral load. Mice that did not receive prophylactic treatment but were only treated post-challenge also showed a statistically significant reduction in lung tissue viral load albeit less than mice that received prophylactic treatment.

RSV-A2 infection in mice resulted in a strong immune response evidenced by changes in BAL fluid cell counts (total cell count, neutrophils and lymphocytes) and cytokines measured in BAL fluid. Groups of mice treated with Neumifil showed statistically significant improvements in a number of immune parameters; these results correlated well with the observed reductions in lung tissue viral loads.

1-7. (canceled)

8. A method of treating and/or preventing a lung inflammatory disease, pneumonia, bronchiolitis and/or bronchitis, said method comprising administering a subject in need thereof, a therapeutically effective amount of a sialic acid binding molecule.

9. The method of claim 8, wherein the sialic acid binding molecule comprises or consists essentially of the sequence of SEQ ID NO: 9 or a sequence at least 85% identical thereto.

10. The method of claim 8, wherein the sialic acid binding molecule comprises or consists essentially of, the following sequence:
GAMVIEKEDVETNASNGQRVDLSSELDKLKKLENATVHMEFKPDPKAPAFYNLFSVSSATKKDEYETMAV
YNNTATLEGRGSDGKQFYNNYNDAPLKVKPGQWNSVTFTVEKPTAELPKGRARLYVNGGLSRTSLRSGNE
IKDMPDVTHVQIGATKRANNTVWGSNLQIRNLTVYNRALTPEEVQKRSGGGSGVIEKEDVETNASNGQRV
DLSSELDKLKKLENATVHMEFKPDPKAPAFYNLFSVSSATKKDEYFTMAVYNNTATLEGRGSDGKQFYNN
YNDAPLKVKPGQWNSVTFTVEKPTAELPKGRARLYVNGGLSRTSLRSGNFIKDMPDVTHVQIGATKRANN
TVWGSNLQIRNLTVYNRALTPEEVQKRSGGSLGVPDFESDWFDVSSNSLYTLSHGLQRSPRRVVVEFARS
SSPSTWNIVMPSYFNDGGHKGSGAQVEVGSLNIKLGTGAAVWGTGYFGGIDNSATTRFATGYYRVRAWI.

11. The method of claim 8, wherein the sialic acid binding molecule is administered intranasally.

12. The method of claim 8, wherein the method comprises mucosally administering a composition comprising the sialic acid binding molecule to the subject in need thereof.

13. The method of claim 8, wherein the method is a method of preventing a lung inflammatory disease, pneumonia, bronchiolitis and/or bronchitis and the sialic acid binding molecule is used prophylactically.

14. The method of claim 8, wherein the sialic acid binding molecule does not exhibit sialidase activity.

15. A method of treating and/or preventing a disease selected from the group consisting of:
(i) inflammatory diseases;
(ii) diseases and/or conditions with an inflammatory aeitiology;
(iii) a lung inflammatory disease;
(iv) inflammation occurring as a consequence of a cascade of cytokines;
(v) pneumonia;
(vi) bronchiolitis; and
(vii) bronchitis;
said method comprising administering to a subject in need thereof, a therapeutically effective amount of a sialic acid binding molecule;
wherein the sialic acid binding molecule comprises, or consists essentially of, the sequence of SEQ ID NO: 9 or a sequence at least 85% identical thereto.


SEQUENCE LISTING (31 sequences)

SEQ ID NO: 1 - protein, 781 aa, Vibrio cholerae
MRFKNVKKTALMLAMFGMATSSNAALFDYNATGDTEFDSPAKQGWMQDNTNNGSGVLTNADGMPAWLVQGIGGRAQWTYSLSTNQHAQASSFGWRMTTEMKVLSGGMITNYYANGTQRVLPIISLDSSGNLVVEFEGQTGRTVLATGTAATEYHKFELVFLPGSNPSASFYFDGKLIRDNIQPTASKQNMIVWGNGSSNTDGVAAYRDIKFEIQGDVIFRGPDRIPSIVASSVTPGVVTAFAEKRVGGGDPGALSNTNDIITRTSRDGGITWDTELNLTEQINVSDEFDFSDPRPIYDPSSNTVLVSYARWPTDAAQNGDRIKPWMPNGIFYSVYDVASGNWQAPIDVTDQVKERSFQIAGWGGSELYRRNTSLNSQQDWQSNAKIRIVDGAANQIQVADGSRKYVVTLSIDESGGLVANLNGVSAPIILQSEHAKVHSFHDYELQYSALNHTTTLFVDGQQITTWAGEVSQENNIQFGNADAQIDGRLHVQKIVLTQQGHNLVEFDAFYLAQQTPEVEKDLEKLGWTKIKTGNTMSLYGNASVNPGPGHGITLTRQQNISGSQNGRLIYPAIVLDRFFLNVMSIYSDDGGSNWQTGSTLPIPFRWKSSSILETLEPSEADMVELQNGDLLLTARLDFNQIVNGVNYSPRQQFLSKDGGITWSLLEANNANVFSNISTGTVDASITRFEQSDGSHFLLFTNPQGNPAGTNGRQNLGLWFSFDEGVTWKGPIQLVNGASAYSDIYQLDSENAIVIVETDNSNMRILRMPITLLKQKLTLSQN

SEQ ID NO: 2 - protein, 192 aa, Vibrio cholerae
ALFDYNATGDTEFDSPAKQGWMQDNTNNGSGVLTNADGMPAWLVQGIGGRAQWTYSLSTNQHAQASSFGWRMTTEMKVLSGGMITNYYANGTQRVLPIISLDSSGNLVVEFEGQTGRTVLATGTAATEYHKFELVFLPGSNPSASFYFDGKLIRDNIQPTASKQNMIVWGNGSSNTDGVAAYRDIKFEIQGD

SEQ ID NO: 3 - protein, 1035 aa, Streptococcus pneumoniae
MSYFRNRDIDIERNSMNRSVQERKCRYSIRKLSVGAVSMIVGAVVFGTSPVLAQEGASEQPLANETQLSGESSTLTDTEKSQPSSETELSGNKQEQERKDKQEEKIPRDYYARDLENVETVIEKEDVETNASNGQRVDLSSELDKLKKLENATVHMEFKPDAKAPAFYNLFSVSSATKKDEYFTMAVYNNTATLEGRGSDGKQFYNNYNDAPLKVKPGQWNSVTFTVEKPTAELPKGRVRLYVNGVLSRTSLRSGNFIKDMPDVTHVQIGATKRANNTVWGSNLQIRNLTVYNRALTPEEVQKRSQLFKRSDLEKKLPEGAALTEKTDIFESGRNGKPNKDGIKSYRIPALLKTDKGTLIAGADERRLHSSDWGDIGMVIRRSEDNGKTWGDRVTITNLRDNPKASDPSIGSPVNIDMVLVQDPETKRIFSIYDMFPEGKGIFGMSSQKEEAYKKIDGKTYQILYREGEKGAYTIRENGTVYTPDGKATDYRVVVDPVKPAYSDKGDLYKGNQLLGNIYFTTNKTSPFRIAKDSYLWMSYSDDDGKTWSAPQDITPMVKADWMKFLGVGPGTGIVLRNGPHKGRILIPVYTTNNVSHLNGSQSSRIIYSDDHGKTWHAGEAVNDNRQVDGQKIHSSTMNNRRAQNTESTVVQLNNGDVKLFMRGLTGDLQVATSKDGGVTWEKDIKRYPQVKDVYVQMSAIHTMHEGKEYIILSNAGGPKRENGMVHLARVEENGELTWLKHNPIQKGEFAYNSLQELGNGEYGILYEHTEKGQNAYTLSFRKFNWDFLSKDLISPTEAKVKRTREMGKGVIGLEFDSEVLVNKAPTLQLANGKTARFMTQYDTKTLLFTVDSEDMGQKVTGLAEGAIESMHNLPVSVAGTKLSNGMNGSEAAVHEVPEYTGPLGTSGEEPAPTVEKPEYTGPLGTSGEEPAPTVEKPEYTGPLGTAGEEAAPTVEKPEFTGGVNGTEPAVHEIAEYKGSDSLVTLTTKEDYTYKAPLAQQALPETGNKESDLLASLGLTAFFLGLFTLGKKREQ

SEQ ID NO: 4 - protein, 185 aa, Streptococcus pneumoniae
VIEKEDVETNASNGQRVDLSSELDKLKKLENATVHMEFKPDAKAPAFYNLFSVSSATKKDEYFTMAVYNNTATLEGRGSDGKQFYNNYNDAPLKVKPGQWNSVTFTVEKPTAELPKGRVRLYVNGVLSRTSLRSGNFIKDMPDVTHVQIGATKRANNTVWGSNLQIRNLTVYNRALTPEEVQKRS

SEQ ID NO: 5 - protein, 438 aa, Pseudomonas aeruginosa
MNTYFDIPHRLVGKALYESYYDHFGQMDILSDGSLYLIYRRATEHVGGSDGRVVFSKLEGGIWSAPTIVAQAGGQDFRDVAGGTMPSGRIVAASTVYETGEVKVYVSDDSGVTWVHKFTLARGGADYNFAHGKSFQVGARYVIPLYAATGVNYELKWLESSDGGETWGEGSTIYSGNTPYNETSYLPVGDGVILAVARVGSGAGGALRQFISLDDGGTWTDQGNVTAQNGDSTDILVAPSLSYIYSEGGTPHVVLLYTNRTTHFCYYRTILLAKAVAGSSGWTERVPVYSAPAASGYTSQVVLGGRRILGNLFRETSSTTSGAYQFEVYLGGVPDFESDWFSVSSNSLYTLSHGLQRSPRRVVVEFARSSSPSTWNIVMPSYFNDGGHKGSGAQVEVGSLNIRLGTGAAVWGTGYFGGIDNSATTRFATGYYRVRAWI

SEQ ID NO: 6 - protein, 106 aa, Pseudomonas aeruginosa
VPDFESDWFSVSSNSLYTLSHGLQRSPRRVVVEFARSSSPSTWNIVMPSYFNDGGHKGSGAQVEVGSLNIRLGTGAAVWGTGYFGGIDNSATTRFATGYYRVRAWI

SEQ ID NO: 7 - protein, 489 aa, Streptococcus pneumoniae
GAMVIEKEDVETNASNGQRVDLSSELDKLKKLENATVHMEFKPDAKAPAFYNLFSVSSATKKDEYFTMAVYNNTATLEGRGSDGKQFYNNYNDAPLKVKPGQWNSVTFTVEKPTAELPKGRVRLYVNGVLSRTSLRSGNFIKDMPDVTHVQIGATKRANNTVWGSNLQIRNLTVYNRALTPEEVQKRSGGGSGVIEKEDVETNASNGQRVDLSSELDKLKKLENATVHMEFKPDAKAPAFYNLFSVSSATKKDEYFTMAVYNNTATLEGRGSDGKQFYNNYNDAPLKVKPGQWNSVTFTVEKPTAELPKGRVRLYVNGVLSRTSLRSGNFIKDMPDVTHVQIGATKRANNTVWGSNLQIRNLTVYNRALTPEEVQKRSGGALGVPDFESDWFSVSSNSLYTLSHGLQRSPRRVVVEFARSSSPSTWNIVMPSYFNDGGHKGSGAQVEVGSLNIRLGTGAAVWGTGYFGGIDNSATTRFATGYYRVRAWI

SEQ ID NO: 8 - protein, 489 aa, Artificial Sequence
  Modified CBM
GAMVIEKEDVETNASNGQRVDLSSELDKLKKLENATVHMEFKPDAKAPAFYNLFSVSSATKKDEYFTMAVYNNTATLEGRGSDGKQFYNNYNDAPLKVKPGQWNSVTFTVEKPTAELPKGRARLYVNGGLSRTSLRSGNFIKDMPDVTHVQIGATKRANNTVWGSNLQIRNLTVYNRALTPEEVQKRSGGGSGVIEKEDVETNASNGQRVDLSSELDKLKKLENATVHMEFKPDAKAPAFYNLFSVSSATKKDEYFTMAVYNNTATLEGRGSDGKQFYNNYNDAPLKVKPGQWNSVTFTVEKPTAELPKGRARLYVNGGLSRTSLRSGNFIKDMPDVTHVQIGATKRANNTVWGSNLQIRNLTVYNRALTPEEVQKRSGGSLGVPDFESDWFDVSSNSLYTLSHGLQRSPRRVVVEFARSSSPSTWNIVMPSYFNDGGHKGSGAQVEVGSLNIKLGTGAAVWGTGYFGGIDNSATTRFATGYYRVRAWI

SEQ ID NO: 9 - protein, 489 aa, Artificial Sequence
  Modified CBM
GAMVIEKEDVETNASNGQRVDLSSELDKLKKLENATVHMEFKPDPKAPAFYNLFSVSSATKKDEYFTMAVYNNTATLEGRGSDGKQFYNNYNDAPLKVKPGQWNSVTFTVEKPTAELPKGRARLYVNGGLSRTSLRSGNFIKDMPDVTHVQIGATKRANNTVWGSNLQIRNLTVYNRALTPEEVQKRSGGGSGVIEKEDVETNASNGQRVDLSSELDKLKKLENATVHMEFKPDPKAPAFYNLFSVSSATKKDEYFTMAVYNNTATLEGRGSDGKQFYNNYNDAPLKVKPGQWNSVTFTVEKPTAELPKGRARLYVNGGLSRTSLRSGNFIKDMPDVTHVQIGATKRANNTVWGSNLQIRNLTVYNRALTPEEVQKRSGGSLGVPDFESDWFDVSSNSLYTLSHGLQRSPRRVVVEFARSSSPSTWNIVMPSYFNDGGHKGSGAQVEVGSLNIKLGTGAAVWGTGYFGGIDNSATTRFATGYYRVRAWI

SEQ ID NO: 10 - protein, 5 aa, Artificial Sequence
  Linker
  MISC_FEATURE (3)..(3): any amino acid
ALXGS

SEQ ID NO: 11 - protein, 5 aa, Artificial Sequence
  Linker
LQALG

SEQ ID NO: 12 - protein, 5 aa, Artificial Sequence
  Linker
  MISC_FEATURE (3)..(3): any amino acid
GGXSG

SEQ ID NO: 13 - protein, 5 aa, Artificial Sequence
  Linker
GGALG

SEQ ID NO: 14 - protein, 5 aa, Artificial Sequence
  Linker
GGGGS

SEQ ID NO: 15 - protein, 10 aa, Artificial Sequence
  Linker
  MISC_FEATURE (3)..(3): Any amino acid
ALXGSGGGSG

SEQ ID NO: 16 - protein, 10 aa, Artificial Sequence
  Linker
LQALGGGGSL

SEQ ID NO: 17 - protein, 15 aa, Artificial Sequence
  Linker
  MISC_FEATURE (3)..(3): Any amino acid
ALXGSGGGSGGGGSG

SEQ ID NO: 18 - protein, 393 aa, Artificial Sequence
  Multivalent CBM
GAMALFDYNATGDTEFDSPAKQGWMQDNTNNGSGVLTNADGMPAWLVQGIGGRAQWTYSLSTNQHAQASSFGWRMTTEMKVLSGGMITNYYANGTQRVLPIISLDSSGNLVVEFEGQTGRTVLATGTAATEYHKFELVFLPGSNPSASFYFDGKLIRDNIQPTASKQNMIVWGNGSSNTDGVAAYRDIKFEIQGDALNGSMALFDYNATGDTEFDSPAKQGWMQDNTNNGSGVLTNADGMPAWLVQGIGGRAQWTYSLSTNQHAQASSFGWRMTTEMKVLSGGMITNYYANGTQRVLPIISLDSSGNLVVEFEGQTGRTVLATGTAATEYHKFELVFLPGSNPSASFYFDGKLIRDNIQPTASKQNMIVWGNGSSNTDGVAAYRDIKFEIQGD

SEQ ID NO: 19 - protein, 789 aa, Artificial Sequence
  Multivalent CBM
GAMALFDYNATGDTEFDSPAKQGWMQDNTNNGSGVLTNADGMPAWLVQGIGGRAQWTYSLSTNQHAQASSFGWRMTTEMKVLSGGMITNYYANGTQRVLPIISLDSSGNLVVEFEGQTGRTVLATGTAATEYHKFELVFLPGSNPSASFYFDGKLIRDNIQPTASKQNMIVWGNGSSNTDGVAAYRDIKFEIQGDALNGSMALFDYNATGDTEFDSPAKQGWMQDNTNNGSGVLTNADGMPAWLVQGIGGRAQWTYSLSTNQHAQASSFGWRMTTEMKVLSGGMITNYYANGTQRVLPIISLDSSGNLVVEFEGQTGRTVLATGTAATEYHKFELVFLPGSNPSASFYFDGKLIRDNIQPTASKQNMIVWGNGSSNTDGVAAYRDIKFEIQGDLQALGMALFDYNATGDTEFDSPAKQGWMQDNTNNGSGVLTNADGMPAWLVQGIGGRAQWTYSLSTNQHAQASSFGWRMTTEMKVLSGGMITNYYANGTQRVLPIISLDSSGNLVVEFEGQTGRTVLATGTAATEYHKFELVFLPGSNPSASFYFDGKLIRDNIQPTASKQNMIVWGNGSSNTDGVAAYRDIKFEIQGDGGNSGMALFDYNATGDTEFDSPAKQGWMQDNTNNGSGVLTNADGMPAWLVQGIGGRAQWTYSLSTNQHAQASSFGWRMTTEMKVLSGGMITNYYANGTQRVLPIISLDSSGNLVVEFEGQTGRTVLATGTAATEYHKFELVFLPGSNPSASFYFDGKLIRDNIQPTASKQNMIVWGNGSSNTDGVAAYRDIKFEIQGD

SEQ ID NO: 20 - protein, 188 aa, Artificial Sequence
  Multivalent CBM
GSMVIEKEDVETNASNGQRVDLSSELDKLKKLENATVHMEFKPDAKAPAFYNLFSVSSATKKDEYFTMAVYNNTATLEGRGSDGKQFYNNYNDAPLKVKPGQWNSVTFTVEKPTAELPKGRVRLYVNGVLSRTSLRSGNFIKDMPDVTHVQIGATKRANNTVWGSNLQIRNLTVYNRALTPEEVQKRS

SEQ ID NO: 21 - protein, 10 aa, Streptococcus pneumoniae
GVLSRTSLRS

SEQ ID NO: 22 - protein, 10 aa, Pseudomonas aeruginosa
WFSVSSNSLY

SEQ ID NO: 23 - protein, 9 aa, Pseudomonas aeruginosa
LSHGLQRSP

SEQ ID NO: 24 - protein, 9 aa, Pseudomonas aeruginosa
GSLNIRLGT

SEQ ID NO: 25 - protein, 12 aa, Streptococcus pneumoniae
FYNLFSVSSATK

SEQ ID NO: 26 - protein, 13 aa, Streptococcus pneumoniae
VRLYVNGVLSRTS

SEQ ID NO: 27 - protein, 15 aa, Streptococcus pneumoniae
KGRVRLYVNGVLSRT

SEQ ID NO: 28 - protein, 15 aa, Pseudomonas aeruginosa
GAQVEVGSLNIRLGT

SEQ ID NO: 29 - protein, 15 aa, Streptococcus pneumoniae
FYNLFSVSSATKKDE

SEQ ID NO: 30 - protein, 15 aa, Pseudomonas aeruginosa
SDWFSVSSNSLYTLS

SEQ ID NO: 31 - protein, 9 aa, Streptococcus pneumoniae
IRNLTVYNR